(19) United States
(12) Patent Application Publication
(10) Pub. No.: US 2025/0215101 A1
(43) Pub. Date: Jul. 3, 2025

(54) DR5 Agonist and PLK1 Inhibitor or CDK Inhibitor Combination Therapy

(71) Applicant: Inhibrx Biosciences, Inc., La Jolla, CA (US)

(72) Inventors: William Crago, San Diego, CA (US); Brendan P. Eckelman, Encinitas, CA (US); Katelyn M. Willis, San Diego, CA (US)

(73) Assignee: Inhibrx Biosciences, Inc., La Jolla, CA (US)

(21) Appl. No.: 18/853,278

(22) PCT Filed: Apr. 7, 2023
(86) PCT No.: PCT/US2023/065489

§ 371 Date: Oct. 1, 2024

Related U.S. Application Data
(60) Provisional application No. 63/328,951, filed on Apr. 8, 2022.

(51) Int. Cl. C07K 16/28; A61K 31/416; A61K 31/519; A61K 38/07; A61K 45/06; A61P 35/00

(52) U.S. Cl. CPC C07K 16/2878; A61K 31/416; A61K 31/519; A61K 38/07; A61K 45/06; A61P 35/00; C07K 2317/22; C07K 2317/35; C07K 2317/565

(57) ABSTRACT

Provided herein are methods of treating cancer with a combination of a DR5 agonist and a PLK1 inhibitor. Also provided herein are methods of treating cancer with a combination of a DR5 agonist and a CDK inhibitor.

### CROSS-REFERENCE TO RELATED APPLICATIONS

This application claims the benefit of priority of U.S. Provisional Application No. 63/328,951, filed Apr. 8, 2022, which is incorporated by reference herein in its entirety for any purpose.

### FIELD

The present invention relates to treatment of cancer with a combination of a DR5 agonist and a PLK1 inhibitor, or the combination of a DR5 agonist and a CDK inhibitor, such as a CDK9 inhibitor.

### BACKGROUND

Death Receptor 5 (DR5; also known as TNFRSF10B or TRAILR2) is a member of the TNF receptor superfamily (TNFRSF) and a cell surface receptor of the TNF-receptor superfamily that binds TNF-related apoptosis-inducing ligand (TRAIL). TRAIL evolved to play critical roles in mammalian development and host defense by selectively eradicating unwanted, infected and malignant cells from healthy cell populations. On binding the TNF receptor family members DR4 or DR5, TRAIL induces cell death via caspase-dependent apoptosis. DR5 appears to be the primary receptor on tumor cells that facilitates the observed tumor biased activity of the TRAIL pathway. DR5 is activated by the natural ligand TRAIL, which brings three DR5 receptors within close proximity thereby activating intracellular caspase-8 and initiating activation of other death-inducing caspases, such as caspases-9 and caspases-3. Thus, initiation of this cell death pathway requires clustering of DR5 receptors for efficient cell death. DR5 agonists are promising therapeutic candidates for treating cancer.

Polo-like kinases belong to the family of mitotic serine/threonine kinases involved in cell cycle progression, the centrosome cycle, mitosis, and cellular response to DNA damage. To date, five polo-like kinases have been identified in mammalian cells: PLK1, PLK2, PLK3, PLK4, and PLK5. Polo-like kinases are highly conserved in many eukaryotic cells and are characterized by an N-terminal serine/threonine protein kinase domain (except PLK5) and the presence of one or two C-terminal regions of similarity called the polo boxes (PB). Out of the polo-like kinase family members, PLK1 is the best characterized. PLK1 plays a role in different stages of cell cycle progression and mitosis including mitotic entry, G2/M checkpoint, spindle assembly maturation, chromosome segregation, and mitotic exit. PLK1 is overexpressed in several tumor types and appears to lead to enhanced proliferation. It is almost undetectable in most normal adult tissue. PLK1 inhibition halts cellular proliferation and causes mitotic catastrophe, making it a therapeutic target for treating cancer.

Cyclin-dependent kinases (CDKs) are serine/threonine protein kinases activated by regulatory cyclin proteins. The human genome encodes twenty CDKs, which play specific roles in numerous cellular processes such as cell division and transcription, in response to intra- and extra-cellular signals. CDK9 is a regulator of transcription, and is harnessed by certain cancer cells for the constant production of proteins to maintain cancer cell survival. In conjunction with its main cyclin partner, Cyclin T1, CDK9 forms Positive Transcription Elongation Factor b (P-TEFb) whose primary function in eukaryotic cells is to mediate transcription elongation of nascent mRNA strands, by phosphorylating the S2 residues of the YSPTSPS tandem repeats at the C-terminal domain (CTD) of RNA Polymerase II (RNAP II). CDK9 is necessary for the expression of myeloid cell leukemia-1 (MCL-1). Dysregulation of the CDK9 pathway has been implicated in the prognosis and resistance to anti-cancer therapeutics in a large number of cancer types, making it a therapeutic target for treating cancer.

### SUMMARY

Provided herein are methods of treating cancer in a subject with a Death Receptor 5 (DR5) agonist and a Polo-Like Kinase 1 (PLK1) inhibitor. In some embodiments, the methods comprise administering a multivalent Death Receptor 5 (DR5)-binding polypeptide and a Polo-Like Kinase 1 (PLK1) inhibitor. Also provided herein are methods of treating cancer in a subject with a Death Receptor 5 (DR5) agonist and a Cyclin-dependent kinase 9 (CDK9) inhibitor. In some embodiments, the methods comprise administering a multivalent Death Receptor 5 (DR5)-binding polypeptide and a CDK9 inhibitor. In some embodiment, the multivalent DR5-binding polypeptide is at least tetravalent. In various embodiments, the DR5-binding polypeptide is a DR5 agonist.

Embodiment 1. A method of treating cancer in a subject in need thereof, comprising administering to the subject (a) a Death Receptor 5 (DR5) agonist, and (b) a Polo-Like Kinase 1 (PLK1) inhibitor.
Embodiment 2. The method of embodiment 1, wherein the DR5 agonist is INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), or MM201-b (Merrimack Pharmaceuticals).
Embodiment 3. The method of embodiment 2, wherein the DR5 agonist is INBRX-109.
Embodiment 4. The method of embodiment 1, wherein the DR5 agonist is a DR5-binding polypeptide.
Embodiment 5. The method of embodiment 4, wherein the DR5-binding polypeptide is at least tetravalent.
Embodiment 6. The method of any one of embodiments 3-5, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 7. The method of embodiment 6, wherein the at least one VHH domain comprises an amino acid sequence at least 90%, at least 95%, at least 99%, or 100% identical to the amino acid sequence of SEQ ID NO: 4.
Embodiment 8. The method of any one of embodiments 3-7, wherein the DR5-binding polypeptide comprises a VHH domain comprising the amino acid sequence of SEQ ID NO: 4.
Embodiment 9. The method of any one of embodiments 3-8, wherein the DR5-binding polypeptide comprises an Fc region.
Embodiment 10. The method of embodiment 9, wherein the Fc region comprises the amino acid sequence of SEQ ID NO: 6.
Embodiment 11. The method of any one of embodiments 3-10, wherein the DR5-binding polypeptide has the structure VHH-linker-VHH-linker-Fc.
Embodiment 12. The method of any one of embodiments 3-11, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 13. The method of any one of embodiments 3-12, wherein the VHH-linker-VHH comprises an amino acid sequence at least 90%, at least 95%, at least 99%, or 100% identical to the amino acid sequence of SEQ ID NO: 5.
Embodiment 14. The method of embodiment 13, wherein the VHH-linker-VHH comprises the amino acid sequence of SEQ ID NO: 5.
Embodiment 15. The method of any one of embodiments 3-14, wherein the DR5-binding polypeptide comprises an amino acid sequence at least 90%, at least 95%, at least 99%, or 100% identical to the amino acid sequence of SEQ ID NO: 7.
Embodiment 16. The method of any one of embodiments 3-15, wherein the DR5-binding polypeptide comprises the amino acid sequence of SEQ ID NO: 7.
Embodiment 17. The method of any one of embodiments 3-15, wherein the DR5-binding polypeptide consists of the amino acid sequence of SEQ ID NO: 7.
Embodiment 18. The method of any one of embodiments 1-17, wherein the PLK1 inhibitor is a small molecule or an interfering RNA (siRNA).
Embodiment 19. The method of any one of embodiments 1-18, wherein the PLK1 inhibitor is onvansertib, volasertib, rigosertib, B12536 (Boehringer Ingelheim), N-[[4-[(6-Chloro-3-pyridinyl)methoxy]-3-methoxyphenyl]methyl]-3,4-dimethoxybenzeneethanamine hydrochloride (SBE 13 HCl), MLN0905 (Takeda Oncology), GSK461364 (GlaxosSmithKline), poloxin, poloxin-2HT, RO3280 (CAS No. 1062243-51-9), HMN-214 (CAS No. 173529-46-9), HMN-176 (CAS No. 173529-10-7), 2-cyano-2-[3-ethyl-4-oxo-5-[[3-(2-pyrrolidin-1-ylethyl)anilino]methyl]-1,3-thiazolidin-2-yl]-N-(2,2,2-trifluoroethyl)acetamide (ZK-thiazolidinone), or cyclapolin 9 (CAS No. 40533-25-3), 5-(5,6-Dimethoxy-1H-benzimidazol-1-yl)-3-[[2-(trifluoromethyl)phenyl]methoxy]-2-thiophenecarboxamide (GW 843682X).
Embodiment 20. The method of embodiment 19, wherein the PLK1 inhibitor is onvansertib, volasertib, rigosertib, B12536 (Boehringer Ingelheim), MLN0905 (Takeda Oncology), GSK461364 (GlaxosSmithKline), CYC140 (Cyclacel), TKM-080301 (TKM-PLK1; Arbutus Biopharma), or TAK-960 (Takeda Pharmaceutical Company).
Embodiment 21. The method of any one of embodiments 1-18, wherein the PLK1 inhibitor is onvansertib.
Embodiment 22. The method of any one of embodiments 1-21, wherein the DR5 agonist and the PLK1 inhibitor are administered separately.
Embodiment 23. The method of embodiment 22, wherein the DR5 agonist and the PLK1 inhibitor are administered sequentially.
Embodiment 24. The method of embodiment 22 or 23, wherein at least one dose, or the first dose, of the DR5 agonist is administered before the PLK1 inhibitor.
Embodiment 25. The method of embodiment 22 or 23, wherein at least one dose, or the first dose, of the DR5 agonist is administered after the PLK1 inhibitor.
Embodiment 26. The method of any one of embodiments 1-21, wherein the DR5 agonist and the PLK1 inhibitor are administered concurrently.
Embodiment 27. The method of any one of embodiments 1-26, wherein the DR5 agonist and the PLK1 inhibitor act synergistically.
Embodiment 28. The method of embodiment 27, wherein synergy is determined in an in vitro cell survival assay.
Embodiment 29. The method of any one of embodiments 1-28, wherein administration of the DR5 agonist and the PLK1 inhibitor results in a synergistic effect, compared to each agent administered alone.
Embodiment 30. A method of treating cancer in a subject in need thereof, comprising administering to the subject (a) a Death Receptor 5 (DR5) agonist, and (b) a Cyclin-Dependent Kinase (CDK) inhibitor.
Embodiment 31. The method of embodiment 30, wherein the DR5 agonist is INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), or MM201-b (Merrimack Pharmaceuticals).
Embodiment 32. The method of embodiment 31, wherein the DR5 agonist is INBRX-109.
Embodiment 33. The method of embodiment 30, wherein the DR5 agonist is a DR5-binding polypeptide.
Embodiment 34. The method of embodiment 33, wherein the DR5-binding polypeptide is at least tetravalent.
Embodiment 35. The method of any one of embodiments 32-34, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 36. The method of embodiment 35, wherein the at least one VHH domain comprises an amino acid sequence at least 90%, at least 95%, at least 99%, or 100% identical to the amino acid sequence of SEQ ID NO: 4.
Embodiment 37. The method of any one of embodiments 32-36, wherein the DR5-binding polypeptide comprises a VHH domain comprising the amino acid sequence of SEQ ID NO: 4.
Embodiment 38. The method of any one of embodiments 32-37, wherein the DR5-binding polypeptide comprises an Fc region.
Embodiment 39. The method of embodiment 38, wherein the Fc region comprises the amino acid sequence of SEQ ID NO: 6.
Embodiment 40. The method of any one of embodiments 32-39, wherein the DR5-binding polypeptide has the structure VHH-linker-VHH-linker-Fc.
Embodiment 41. The method of any one of embodiments 32-40, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 42. The method of any one of embodiments 32-41, wherein the VHH-linker-VHH comprises an amino acid sequence at least 90%, at least 95%, at least 99%, or 100% identical to the amino acid sequence of SEQ ID NO: 5.
Embodiment 43. The method of embodiment 42, wherein the VHH-linker-VHH comprises the amino acid sequence of SEQ ID NO: 5.
Embodiment 44. The method of any one of embodiments 32-43, wherein the DR5-binding polypeptide comprises an amino acid sequence at least 90%, at least 95%, at least 99%, or 100% identical to the amino acid sequence of SEQ ID NO: 7.
Embodiment 45. The method of any one of embodiments 32-44, wherein the DR5-binding polypeptide comprises the amino acid sequence of SEQ ID NO: 7.
Embodiment 46. The method of any one of embodiments 32-44, wherein the DR5-binding polypeptide consists of the amino acid sequence of SEQ ID NO: 7.
Embodiment 47. The method of any one of embodiments 30-46, wherein the CDK inhibitor is a CDK9 inhibitor.
Embodiment 48. The method of any one of embodiments 30-47, wherein the CDK inhibitor is a small molecule.
Embodiment 49. The method of any one of embodiments 30-48, wherein the CDK inhibitor is flavopiridol, seliciclib, dinaciclib, atuveciclib, enitociclib, AZD4573, i-CDK9, or NVP-2.
Embodiment 50. The method of any one of embodiments 49, wherein the CDK inhibitor is dinaciclib, NVP-2, flavopiridol, enitociclib, or AZD4573.
Embodiment 51. The method of any one of embodiments 30-50, wherein the DR5 agonist and the CDK inhibitor are administered separately.
Embodiment 52. The method of embodiment 51, wherein the DR5 agonist and the CDK inhibitor are administered sequentially.
Embodiment 53. The method of embodiment 51 or 52, wherein at least one dose, or the first dose, of the DR5 agonist is administered before the CDK inhibitor.
Embodiment 54. The method of embodiment 51 or 52, wherein at least one dose, or the first dose, of the DR5 agonist is administered after the CDK inhibitor.
Embodiment 55. The method of any one of embodiments 30-51, wherein the DR5 agonist and the CDK inhibitor are administered concurrently.
Embodiment 56. The method of any one of embodiments 30-55, wherein the DR5 agonist and the CDK inhibitor act synergistically.
Embodiment 57. The method of embodiment 56, wherein synergy is determined in an in vitro cell survival assay.
Embodiment 58. The method of any one of embodiments 30-57, wherein administration of the DR5 agonist and the CDK inhibitor results in a synergistic effect, compared to each agent administered alone.
Embodiment 59. The method of any one of embodiments 1-58, wherein the cancer is adrenal cancer; astrocytoma; basal cell carcinoma, biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and/or pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.
Embodiment 60. A DR5 agonist for use in a method of treating cancer, wherein the method comprises administering the DR5 agonist in combination with a PLK1 inhibitor.
Embodiment 61. Use of a DR5 agonist for the manufacture of a medicament for treating cancer, wherein the medicament is for administration with a PLK1 inhibitor.
Embodiment 62. A DR5 agonist for use in a method of treating cancer, wherein the method comprises administering the DR5 agonist in combination with a CDK inhibitor, such as a CDK9 inhibitor.
Embodiment 63. Use of a DR5 agonist for the manufacture of a medicament for treating cancer, wherein the medicament is for administration with a CDK inhibitor, such as a CDK9 inhibitor.

### BRIEF DESCRIPTION OF THE DRAWINGS

FIG. 1A-1F shows results of a titration experiment in which different cancer cell lines were contacted with varying concentrations of INBRX-109 (0.00001, 0.0001, 0.001, 0.01, 0.1, 1, or 10 nM) and onvansertib (0.32, 1.6, 8, 40, 200, or 1000 nM). Percent survival of cancer cells is shown on the y-axis of each graph. The results are shown for cancer cell lines HT-29 (FIG. 1A), LS174T (FIG. 1B), SW620 (FIG. 1C), SW837 (FIG. 1D), SW1463 (FIG. 1E), and LS411N (FIG. 1F).

FIG. 2A-2F shows results of an INBRX-109 titration experiment in which different cancer cell lines were contacted with varying concentrations of INBRX-109 (0.0001, 0.001, 0.01, 0.1, 1, or 10 nM), alone or in combination with 200 nM onvansertib. Percent survival of cancer cells is shown on the y-axis of each graph. The results are shown for cancer cell lines HT-29 (FIG. 2A), LS 174T (FIG. 2B), SW620 (FIG. 2C), SW837 (FIG. 2D), SW1463 (FIG. 2E), and LS411N (FIG. 2F). The dotted line labeled “Cpd alone” shows the percent survival of cancer cells treated with 200 nM onvansertib alone.

FIG. 3A-3F shows results of an onvansertib titration experiment in which different cancer cell lines were contacted with varying concentrations of onvansertib (0.32, 1.6, 8, 40, 200, or 1000 nM), alone or in combination with 1 nM INBRX-109. Percent survival of cancer cells is shown on the y-axis of each graph. The results are shown for cancer cell lines HT-29 (FIG. 3A), LS174T (FIG. 3B), SW620 (FIG. 3C), SW837 (FIG. 3D), SW1463 (FIG. 3E), and LS411N (FIG. 3F). The dotted line labeled “Ab alone” shows the percent survival of cancer cells treated with 1 nM INBRX-109 alone.

FIGS. 4A-4I, 5A-5I, and 6A-6L show the results of an onvansertib titration experiment in which different colorectal cancer cell lines were contacted with varying concentrations of onvansertib (274 pM, 823 pM, 2.47 nM, 7.41 nM, 22.22 nM, 66.67 nM, 200 nM, 600 nM, and 1.8 μM), alone or in combination with 1 nM INBRX-109. The results are shown for cancer cell lines SK-CO-1 (FIG. 4A), SNU-C5 (FIG. 4B), LS 1034 (FIG. 4C), LS 123 (FIG. 4D), LS 180 (FIG. 4E), T84 (FIG. 4F), SW-48 (FIG. 4G), SW01116 (FIG. 411), SW-1463 (FIG. 4I), SW-837 (FIG. 5A), SW-948 (FIG. 5B), LS 513 (FIG. 5C), NCI-H716 (FIG. 5D), HCT 116 (FIG. 5E), DLD-1 (FIG. 5F), HCT-15 (FIG. 5G), LS 174T (FIG. 511), Ht-55 (FIG. 5I), SNU-81 (FIG. 6A), HT-29 (FIG. 6B), SW-620 (FIG. 6C), Hu Tu 80 (FIG. 6D), SW-480 (FIG. 6E), SW-403 (FIG. 6F), LoVo (FIG. 6G), SNU-C2A (FIG. 611), NCI-H747 (FIG. 6I), COLO320DM (FIG. 6J), RKO (FIG. 6K), HCT-8 (FIG. 6L). Percent survival of cancer cells is shown on the y-axis of each graph. The dotted line labeled “Ab alone” shows the percent survival of cancer cells treated with 1 nM INBRX-109 alone.

FIGS. 7A-7I and 8A-811 show the results of an onvansertib titration experiment in which different pancreatic cancer cell lines were contacted with varying concentrations of onvansertib (274 pM, 823 pM, 2.47 nM, 7.41 nM, 22.22 nM, 66.67 nM, 200 nM, 600 nM, and 1.8 μM), alone or in combination with 1 nM INBRX-109. The results are shown for cancer cell lines MA PaCa-2 (FIG. 7A), Panc 05.24 (FIG. 7B), SW-1990 (FIG. 7C), HuP-T4 (FIG. 7D), BxPC-3 (FIG. 7E), Capan-2 (FIG. 7F), KP4 (FIG. 7G), AsPC-1 (FIG. 711), PSN-1 (FIG. 7I), HPAF-II (FIG. 8A), PANC-1 (FIG. 8B), Capan-1 (FIG. 8C), CFPAC-1 (FIG. 8D), Panc 03.27 (FIG. 8E), SU.86.86 (FIG. 8F), PL45 (FIG. 8G), Panc 10.05 (FIG. 811). Percent survival of cancer cells is shown on the y-axis of each graph. The dotted line labeled “Ab alone” shows the percent survival of cancer cells treated with 1 nM INBRX-109 alone.

FIG. 9A-9E show results of a titration experiment in which different cancer cell lines were contacted with varying concentrations of INBRX-109 (0.00001, 0.0001, 0.001, 0.01, 0.1, 1, or 10 nM) and dinaciclib (0.2, 1, 5, 25, 125, or 625 nM). Percent survival of cancer cells is shown on the y-axis of each graph. The results are shown for cancer cell lines CAL-78 (FIG. 9A), OUMS-27 (FIG. 9B), SW1353 (FIG. 9C), H-EMC-SS (FIG. 9D), and SW620 (FIG. 9E).

FIG. 10A-10E show results of an INBRX-109 titration experiment in which different cancer cell lines were contacted with varying concentrations of INBRX-109 (0.0001, 0.001, 0.01, 0.1, 1, or 10 nM), alone or in combination with 25 nM dinaciclib. Percent survival of cancer cells is shown on the y-axis of each graph. The results are shown for cancer cell lines CAL-78 (FIG. 10A), OUMS-27 (FIG. 10B), SW1353 (FIG. 10C), H-EMC-SS (FIG. 10D), and SW620 (FIG. 10E). The dotted line labeled “Cpd alone” shows the percent survival of cancer cells treated with 25 nM dinaciclib alone.

FIG. 11A-11E show results of a dinaciclib titration experiment in which different cancer cell lines were contacted with varying concentrations of dinaciclib (0.2, 1, 5, 25, 125, or 625 nM), alone or in combination with 1 nM INBRX-109. Percent survival of cancer cells is shown on the y-axis of each graph. The results are shown for cancer cell lines CAL-78 (FIG. 11A), OUMS-27 (FIG. 11B), SW1353 (FIG. 11C), H-EMC-SS (FIG. 11D), and SW620 (FIG. 11E). The dotted line labeled “Ab alone” shows the percent survival of cancer cells treated with 1 nM INBRX-109 alone.

FIG. 12A-12E show results of a titration experiment in which different cancer cell lines were contacted with varying concentrations of INBRX-109 (0.00001, 0.0001, 0.001, 0.01, 0.1, 1, or 10 nM) and NVP-2 (0.2, 1, 5, 25, 125, or 625 nM). Percent survival of cancer cells is shown on the y-axis of each graph. The results are shown for cancer cell lines CAL-78 (FIG. 12A), OUMS-27 (FIG. 12B), SW1353 (FIG. 12C), H-EMC-SS (FIG. 12D), and SW620 (FIG. 12E).

FIG. 13A-13E show results of an INBRX-109 titration experiment in which different cancer cell lines were contacted with varying concentrations of INBRX-109 (0.0001, 0.001, 0.01, 0.1, 1, or 10 nM), alone or in combination with 125 nM NVP-2. Percent survival of cancer cells is shown on the y-axis of each graph. The results are shown for cancer cell lines CAL-78 (FIG. 13A), OUMS-27 (FIG. 13B), SW1353 (FIG. 13C), H-EMC-SS (FIG. 13D), and SW620 (FIG. 13E). The dotted line labeled “Cpd alone” shows the percent survival of cancer cells treated with 125 nM NVP-2 alone.

FIG. 14A-14E show results of an NVP-2 titration experiment in which different cancer cell lines were contacted with varying concentrations of NVP-2 (0.2, 1, 5, 25, 125, or 625 nM), alone or in combination with 1 nM INBRX-109. Percent survival of cancer cells is shown on the y-axis of each graph. The results are shown for cancer cell lines CAL-78 (FIG. 14A), OUMS-27 (FIG. 14B), SW1353 (FIG. 14C), H-EMC-SS (FIG. 14D), and SW620 (FIG. 14E). The dotted line labeled “Ab alone” shows the percent survival of cancer cells treated with 1 nM INBRX-109 alone.

FIG. 15A-15F show results of a titration experiment in which different cancer cell lines were contacted with varying concentrations of INBRX-109 (0.00001, 0.0001, 0.001, 0.01, 0.1, 1, or 10 nM) and flavopiridol (0.2, 1, 5, 25, 125, or 625 nM). Percent survival of cancer cells is shown on the y-axis of each graph. The results are shown for cancer cell lines CAL-78 (FIG. 15A), OUMS-27 (FIG. 15B), HS-SY-II (FIG. 15C), SW1353 (FIG. 15D), H-EMC-SS (FIG. 15E) and SW620 (FIG. 15F).

FIG. 16A-16F show results of an INBRX-109 titration experiment in which different cancer cell lines were contacted with varying concentrations of INBRX-109 (0.0001, 0.001, 0.01, 0.1, 1, or 10 nM), alone or in combination with 125 nM flavopiridol. Percent survival of cancer cells is shown on the y-axis of each graph. The results are shown for cancer cell lines CAL-78 (FIG. 16A), OUMS-27 (FIG. 16B), HS-SY-II (FIG. 16C), SW1353 (FIG. 16D), H-EMC-SS (FIG. 16E) and SW620 (FIG. 16F). The dotted line labeled “Cpd alone” shows the percent survival of cancer cells treated with 125 nM flavopiridol alone.

FIG. 17A-17F show results of an flavopiridol titration experiment in which different cancer cell lines were contacted with varying concentrations of flavopiridol (0.2, 1, 5, 25, 125, or 625 nM), alone or in combination with 1 nM INBRX-109. Percent survival of cancer cells is shown on the y-axis of each graph. The results are shown for cancer cell lines CAL-78 (FIG. 17A), OUMS-27 (FIG. 17B), HS-SY-II (FIG. 17C), SW1353 (FIG. 17D), H-EMC-SS (FIG. 17E) and SW620 (FIG. 17F). The dotted line labeled “Ab alone” shows the percent survival of cancer cells treated with 1 nM INBRX-109 alone.

FIG. 18A-18F show results of a titration experiment in which different cancer cell lines were contacted with varying concentrations of INBRX-109 (0.00001, 0.0001, 0.001, 0.01, 0.1, 1, or 10 nM) and enitociclib (0.2, 1, 5, 25, 125, or 625 nM). Percent survival of cancer cells is shown on the y-axis of each graph. The results are shown for cancer cell lines CAL-78 (FIG. 18A), OUMS-27 (FIG. 18B), HS-SY-II (FIG. 18C), SW1353 (FIG. 18D), H-EMC-SS (FIG. 18E) and SW620 (FIG. 18F).

FIG. 19A-19F show results of an INBRX-109 titration experiment in which different cancer cell lines were contacted with varying concentrations of INBRX-109 (0.0001, 0.001, 0.01, 0.1, 1, or 10 nM), alone or in combination with 125 nM enitociclib. Percent survival of cancer cells is shown on the y-axis of each graph. The results are shown for cancer cell lines CAL-78 (FIG. 19A), OUMS-27 (FIG. 19B), HS-SY-II (FIG. 19C), SW1353 (FIG. 19D), H-EMC-SS (FIG. 19E) and SW620 (FIG. 19F). The dotted line labeled “Cpd alone” shows the percent survival of cancer cells treated with 125 nM enitociclib alone.

FIG. 20A-20F show results of an enitociclib titration experiment in which different cancer cell lines were contacted with varying concentrations of enitociclib (0.2, 1, 5, 25, 125, or 625 nM), alone or in combination with 1 nM INBRX-109. Percent survival of cancer cells is shown on the y-axis of each graph. The results are shown for cancer cell lines CAL-78 (FIG. 20A), OUMS-27 (FIG. 20B), HS-SY-II (FIG. 20C), SW1353 (FIG. 20D), H-EMC-SS (FIG. 20E) and SW620 (FIG. 20F). The dotted line labeled “Ab alone” shows the percent survival of cancer cells treated with 1 nM INBRX-109 alone.

FIG. 21A-21B shows results of a titration experiment in which different cancer cell lines were contacted with varying concentrations of INBRX-109 (0.00001, 0.0001, 0.001, 0.01, 0.1, 1, or 10 nM) and AZD-5991 (0.0032, 0.016, 0.08, 0.4, 2.0, 10 nM). Percent survival of cancer cells is shown on the y-axis of each graph. The results are shown for cancer cell lines CAL-78 (FIG. 21A), OUMS-27 (FIG. 21B), HS-SY-II (FIG. 21C), SW1353 (FIG. 21D), H-EMC-SS (FIG. 21E) and SW620 (FIG. 21F).

FIG. 22A-22F shows results of an INBRX-109 titration experiment in which different cancer cell lines were contacted with varying concentrations of INBRX-109 (0.0001, 0.001, 0.01, 0.1, 1, or 10 nM), alone or in combination with 2 nM AZD-5991. Percent survival of cancer cells is shown on the y-axis of each graph. The results are shown for cancer cell lines CAL-78 (FIG. 22A), OUMS-27 (FIG. 22B), HS-SY-II (FIG. 22C), SW1353 (FIG. 22D), H-EMC-SS (FIG. 22E) and SW620 (FIG. 22F). The dotted line labeled “Cpd alone” shows the percent survival of cancer cells treated with 2 nM AZD-5991 alone.

FIG. 23A-23F shows results of an AZD-5991 titration experiment in which different cancer cell lines were contacted with varying concentrations of AZD-5991 (0.0032, 0.016, 0.08, 0.4, 2.0, 10 nM), alone or in combination with 1 nM INBRX-109. Percent survival of cancer cells is shown on the y-axis of each graph. The results are shown for cancer cell lines CAL-78 (FIG. 23A), OUMS-27 (FIG. 23B), HS-SY-II (FIG. 23C), SW1353 (FIG. 23D), H-EMC-SS (FIG. 23E) and SW620 (FIG. 23F). The dotted line labeled “Ab alone” shows the percent survival of cancer cells treated with 1 nM INBRX-109 alone.

### DETAILED DESCRIPTION

Embodiments provided herein relate to methods of treating cancer with a combination of a Death Receptor 5 (DR5) agonist and a Polo-Like Kinase 1 (PLK1) inhibitor and/or methods of treating cancer with a combination of a Death Receptor 5 (DR5) agonist and a cyclin-dependent kinase (CDK) inhibitor, such as a CDK9 inhibitor.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., TRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or DR5-related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The terms “DR5,” “death receptor 5,” “TNFRSF10B,” and “TRAILR2” as used herein refer to any native, mature DR5 that results from processing of a DR5 precursor in a cell. The term includes DR5 from any vertebrate source, including mammals such as primates (e.g., humans and cynomolgus or rhesus monkeys) and rodents (e.g., mice and rats), unless otherwise indicated. The term also includes naturally-occurring variants of DR5, such as splice variants or allelic variants. A nonlimiting exemplary precursor human DR5 amino acid sequence is shown, e.g., in NCBI Accession No. NP_003833.4. See SEQ ID NO: 8. A nonlimiting exemplary precursor human DR5 amino acid sequence is shown, e.g., in SEQ ID NO: 9.

The terms “PLK1” and “polo-like kinase 1” as used herein refer to any native, mature PLK1. The term includes PLK1 from any vertebrate source, including mammals such as primates (e.g., humans and cynomolgus or rhesus monkeys) and rodents (e.g., mice and rats), unless otherwise indicated. The term also includes naturally-occurring variants of PLK1, such as splice variants or allelic variants. A nonlimiting PLK1 amino acid sequence is shown, e.g., in UniProtKB/Swiss-Prot Accession No. P53350.1. See SEQ ID NO: 10.

The terms “CDK9” and “cyclin-dependent kinase 9” as used herein refer to any native, mature CDK9. The term includes CDK9 from any vertebrate source, including mammals such as primates (e.g., humans and cynomolgus or rhesus monkeys) and rodents (e.g., mice and rats), unless otherwise indicated. The term also includes naturally-occurring variants of CDK9, such as splice variants or allelic variants. A nonlimiting amino acid sequence is shown, e.g., in UniProtKB/Swiss-Prot Accession No. P50750-1. See SEQ ID NO: 11.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a DR5 epitope is a sdAb or VHH-containing polypeptide that binds this epitope with greater affinity, avidity, more readily, and/or with greater duration than it binds to other DR5 epitopes or non-DR5 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂—VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include F and. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising μ constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation, and inducing or increasing expression of cytokines.

An “agonist” or “activating” antibody or polypeptide is one that increases and/or activates a biological activity of its target antigen. In some embodiments, the agonist antibody or polypeptide binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fe receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGNTM (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: adrenal cancer; astrocytoma; basal cell carcinoma; biliary tract cancer; bladder cancer; bone cancer; brain and central nervous system cancer; breast cancer; cancer of the peritoneum; cervical cancer; choriocarcinoma; chondrosarcoma, Ewing sarcoma, colon and rectum cancer (colorectal cancer); connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The terms “synergistic,” “synergistically,” and “synergy,” as used herein refer to a more than additive effect of two or more agents. A determination of a synergistic effect between a DR5 agonist and a PLK1 inhibitor or between a DR5 agonist and a CDK inhibitor, such as a CDK9 inhibitor may be carried out using the assays described herein.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary DR5 Agonists

Provided herein are methods of treating cancer comprising administering a DR5 agonist. Nonlimiting exemplary DR5 agonists include INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), and MM201-b (Merrimack Pharmaceuticals). In some embodiments, the DR5 agonist is a DR5-binding polypeptide. In some embodiments, a DR5-binding polypeptide provided herein is multivalent. In some embodiments, a DR5-binding polypeptide provided herein is at least tetravalent.

In various embodiments, a DR5-binding polypeptide comprises at least one VHH domain comprising CDR1 comprising the sequence of SEQ ID NO: 1, a CDR2 comprising the sequence of SEQ ID NO: 2, and a CDR3 comprising the sequence of SEQ ID NO: 3. In some embodiments, at least one VHH domain is humanized. In some embodiments, a DR5-binding polypeptide comprises at least one VHH domain comprising an amino acid sequence at least 90%, at least 95%, at least 99%, or 100% identical to the amino acid sequence of SEQ ID NO: 4. In some embodiments, a DR5-binding polypeptide comprises at least one VHH domain comprising the amino acid sequence of SEQ ID NO: 4.

In some embodiments, a DR5-binding polypeptide comprises at least one VHH domain that binds DR5 and an Fc region. In some embodiments, a DR5-binding polypeptide provided herein comprises two VHH domains that bind DR5 and an Fc region. In some embodiments, an Fc region mediates dimerization of the DR5-binding polypeptide at physiological conditions such that a dimer is formed that doubles the number of DR5 binding sites. For example, a DR5-binding polypeptide comprising two VHH domains that bind DR5 and an Fc region is divalent as a monomer, but at physiological conditions, the Fc region may mediate dimerization, such that the DR5-binding polypeptide is a tetravalent dimer under such conditions.

In various embodiments, a DR5-binding polypeptide is provided wherein each VHH domain comprises a CDR1 comprising the sequence of SEQ ID NO: 1, a CDR2 comprising the sequence of SEQ ID NO: 2, and a CDR3 comprising the sequence of SEQ ID NO: 3. In some embodiments, each VHH domain is humanized.

In some embodiments, a DR5-binding polypeptide comprises the structure VHH-linker-VHH-linker-Fc. In some embodiments, the VHH-linker-VHH portion of a DR5-binding polypeptide comprises an amino acid sequence at least 90%, at least 95%, at least 99%, or 100% identical to the amino acid sequence of SEQ ID NO: 5. In some embodiments, the VHH-linker-VHH portion of a DR5-binding polypeptide comprises the amino acid sequence of SEQ ID NO: 5. In some embodiments, the Fc comprises a hinge. In some such embodiments, the Fc comprises the amino acid sequence of SEQ ID NO: 6. In some embodiments, a DR5-binding polypeptide comprises an amino acid sequence at least 90%, at least 95%, at least 99%, or 100% identical to the amino acid sequence of SEQ ID NO: 7, which includes two VHH domains and an Fc region. In some embodiments, a DR5-binding polypeptide comprises the amino acid sequence of SEQ ID NO: 7, which includes two VHH domains and an Fc region. In some embodiments, a DR5-binding polypeptide consists of the amino acid sequence of SEQ ID NO: 7. A DR5-binding polypeptide consisting of the amino acid sequence of SEQ ID NO: 7, or SEQ ID NO: 7 lacking the terminal lysine, may be referred to as INBRX-109.

In some embodiments, a VHH domain that binds DR5 may be humanized.

Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a DR5-binding polypeptide is a human Fc region, or is derived from a human Fc region.

In some embodiments, an Fc region included in a DR5-binding polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling. In some embodiments, the Fc region is a human IgG1 xELL Fc region.

### Exemplary PLK1 Inhibitors

Provided herein are methods of treating cancer comprising administering a PLK1 inhibitor. In some embodiments, the PLK1 inhibitor is a small molecule. In some embodiments, the PLK1 inhibitor is an RNAi. In some embodiments, the PLK1 inhibitor is onvansertib, volasertib, rigosertib, BI2536 (Boehringer Ingelheim), N-[[4-[(6-Chloro-3-pyridinyl)methoxy]-3-methoxyphenyl]methyl]-3,4-dimethoxybenzeneethanamine hydrochloride (SBE 13 HCl), MLN0905 (Takeda Oncology), GSK461364 (GlaxosSmithKline), CYC140 (Cyclacel), TKM-080301 (TKM-PLK1; Arbutus Biopharma), TAK-960 (Takeda Pharmaceutical Company), poloxin, poloxin-2HT, RO3280 (CAS No. 1062243-51-9), 2-cyano-2-[3-ethyl-4-oxo-5-[[3-(2-pyrrolidin-1-ylethyl)anilino]methyl]-1,3-thiazolidin-2-yl]-N-(2,2,2-trifluoroethyl)acetamide (ZK-thiazolidinone), cyclapolin 9 (CAS No. 40533-25-3), 5-(5,6-Dimethoxy-1H-benzimidazol-1-yl)-3-[[2-(trifluoromethyl)phenyl]methoxy]-2-thiophenecarboxamide (GW 843682X), HMN-214 (CAS No. 173529-46-9), or HMN-176 (CAS No. 173529-10-7). In some embodiments, the PLK1 inhibitor is onvansertib, volasertib, rigosertib, BI2536 (Boehringer Ingelheim), MLN0905 (Takeda Oncology), GSK461364 (GlaxosSmithKline), CYC140 (Cyclacel), TKM-080301 (TKM-PLK1; Arbutus Biopharma), or TAK-960 (Takeda Pharmaceutical Company).

In some embodiments, the PLK1 inhibitor is onvansertib. Onvansertib (also known as PCM-075 or NMS-1286937) is a selective ATP-competitive PLK1 inhibitor having the structure

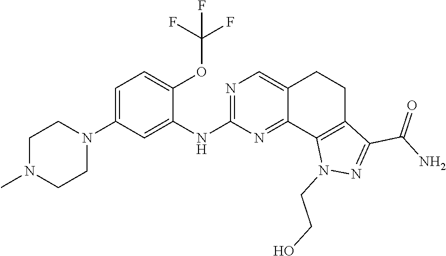 [SMILES: CN1CCN(c2ccc(OC(F)(F)F)c(Nc3ncc4c(n3)-c3c(c(C(N)=O)nn3CCO)CC4)c2)CC1]

or a pharmaceutically acceptable salt or hydrate thereof. See, e.g., U.S. Pat. No. 8,927,530. Onvansertib is specific for PLK1, and has potent in vitro and in vivo antitumor activity in models of both solid and hematologic malignancies.

In some embodiments, the PLK1 inhibitor is volasertib. Volasertib is a selective PLK1 inhibitor having the structure:

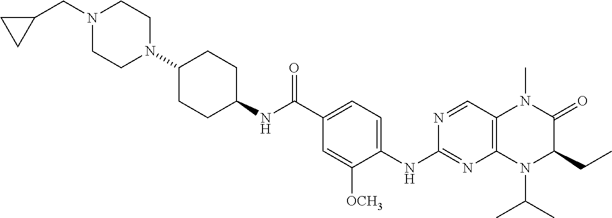

or a pharmaceutically acceptable salt or hydrate thereof. See, e.g., WO 04/076454 and WO 07/090844.

In some embodiments, the PLK1 inhibitor is rigosertib. Rigosertib is an inhibitor of multiple kinases, including PI3-K and PLK1, having the structure:

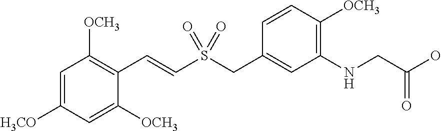 [SMILES: COc1cc(OC)c(/C=C/S(=O)(=O)Cc2ccc(OC)c(NCC(=O)O)c2)c(OC)c1]

or a pharmaceutically acceptable salt or hydrate thereof. See, e.g., U.S. Pat. No. 7,598,232 (compound 4).

In some embodiments, the PLK1 inhibitor is BI2536. BI2536 is a selective inhibitor of PLK1 having the structure:

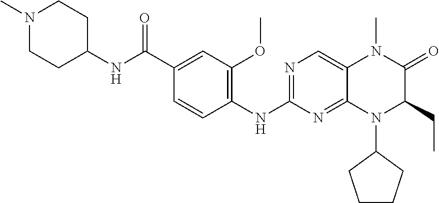 [SMILES: CC[C@@H]1C(=O)N(C)c2cnc(Nc3ccc(C(=O)NC4CCN(C)CC4)cc3OC)nc2N1C1CCCC1]

or a pharmaceutically acceptable salt or hydrate thereof. See, e.g., Steegmaier et al., Current Biology, 17: 316-322 (2007).

In some embodiments, the PLK1 inhibitor is MLN0905. MLN0905 is a selective inhibitor of PLK1 having the structure:

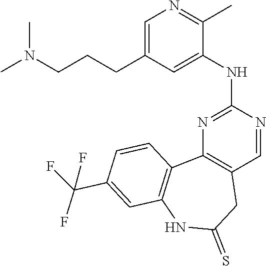 [SMILES: Cc1ncc(CCCN(C)C)cc1Nc1ncc2c(n1)-c1ccc(C(F)(F)F)cc1NC(=S)C2]

S or a pharmaceutically acceptable salt or hydrate thereof. See, e.g., Mol Cancer Ther, 11: 2045-53 (2012).

In some embodiments, the PLK1 inhibitor is TAK-960. TAK-960 is a selective inhibitor of PLK1 having the structure:

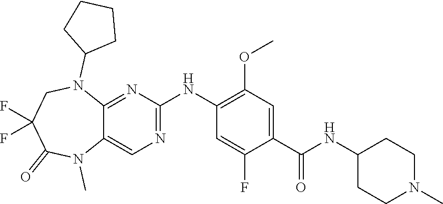 [SMILES: COc1cc(C(=O)NC2CCN(C)CC2)c(F)cc1Nc1ncc2c(n1)N(C1CCCC1)CC(F)(F)C(=O)N2C]

or a pharmaceutically acceptable salt or hydrate thereof. See, e.g., Mol Cancer Ther, 11: 700-9 (2012).

In some embodiments, the PLK1 inhibitor is GSK461364. GSK461364 is an ATP-competitive PLK1 inhibitor having the structure:

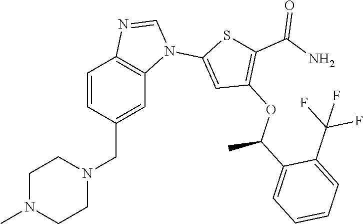 [SMILES: C[C@@H](Oc1cc(-n2cnc3ccc(CN4CCN(C)CC4)cc32)sc1C(N)=O)c1ccccc1C(F)(F)F]

or a pharmaceutically acceptable salt or hydrate thereof. See, e.g., Clin Cancer Res. 17(10):3420-30 (2011).

In some embodiments, the PLK1 inhibitor is CYC140. In some embodiments, the PLK1 inhibitor has the structure:

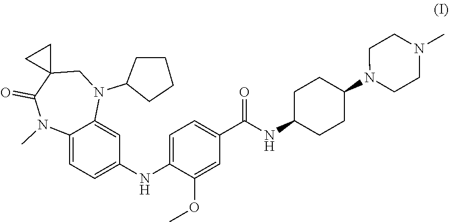 [SMILES: COc1cc(C(=O)N[C@H]2CC[C@@H](N3CCN(C)CC3)CC2)ccc1Nc1ccc2c(c1)N(C1CCCC1)CC1(CC1)C(=O)N2C]

or a pharmaceutically acceptable salt or hydrate thereof. See, e.g., WO 2009/040556.

In some embodiments, the PLK1 inhibitor is TKM-080301. TKM-080301 is a lipid nanoparticle (LNP) formulation comprising four lipids and a synthetic, double-stranded siRNA directed against human PLK1 mRNA. Synthetic siRNAs are a duplex of complementary RNA oligonucleotides designed to achieve post-transcriptional gene suppression through the RNA interference mechanism. See, e.g., Oncologist, 24(6):747-e218 (2019); WO 2008/342535.

### Exemplary CDK Inhibitors

Provided herein are methods of treating cancer comprising administering a CDK inhibitor, such as a CDK9 inhibitor. In some embodiments, the CDK inhibitor is a small molecule. In some embodiments, the CDK inhibitor is flavopiridol (Tolero Pharmaceuticals), seliciclib (roscovitine/CYC202), dinaciclib (Merck), atuveciclib (Bayer), enitociclib (Vincerx Pharma), AZD4573 (AstraZeneca), i-CDK9, or NVP-2.

In some embodiments, the CDK inhibitor is flavopiridol. Flavopiridol (also known as L86-8275, alvocidib, NSC 649890, or HMR-1275; Tolero Pharmaceuticals) is a selective ATP-competitive CDK9 inhibitor having the structure:

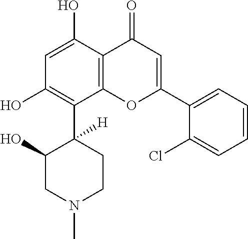 [SMILES: CN1CC[C@H](c2c(O)cc(O)c3c(=O)cc(-c4ccccc4Cl)oc23)[C@H](O)C1]

or a pharmaceutically acceptable salt or hydrate thereof.

Flavopiridol is a potent and selective inhibitor of CDK9 and has antitumor activity against various tumor cells lines, such as human lung carcinoma and breast carcinoma and also inhibits tumor growth in xenograft models. See, e.g., Anshabo, et al., Frontiers in Oncology, 11 (2021).

In some embodiments, the CDK inhibitor is seliciclib (also known as roscovitine or CYC202). Seliciclib is a selective CDK inhibitor having the structure:

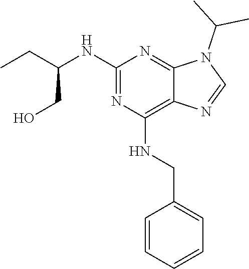 [SMILES: CC[C@H](CO)Nc1nc(NCc2ccccc2)c2ncn(C(C)C)c2n1]

or a pharmaceutically acceptable salt or hydrate thereof. See, e.g., Anshabo, et al., Frontiers in Oncology, 11 (2021).

In some embodiments, the CDK inhibitor is dinaciclib (also known as SCH 727965; Merck). Dinaciclib is a potent, selective small molecule inhibitor of CDKs, including CDK1, CDK2, CDK5, and CDK9, having the structure:

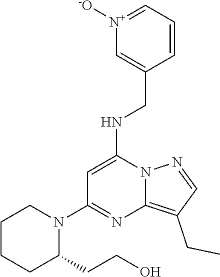

OH or a pharmaceutically acceptable salt or hydrate thereof. See, e.g., Anshabo, et al., Frontiers in Oncology, 11 (2021).

In some embodiments, the CDK inhibitor is atuveciclib (also known as BAY1143572; Bayer). Atuveciclib is a is potent and highly selective inhibitor of positive transcription elongation factor b (PTEF-b), which is composed of CDK9 and cyclin-T (CycT), having the structure:

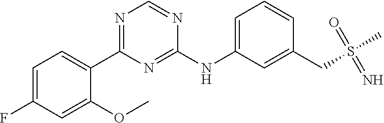 [SMILES: COc1cc(F)ccc1-c1ncnc(Nc2cccc(C[S@](C)(=N)=O)c2)n1]

or a pharmaceutically acceptable salt or hydrate thereof. See, e.g., Anshabo, et al., Frontiers in Oncology, 11 (2021).

In some embodiments, the CDK inhibitor is Enitociclib (also known as BAY1251152 or VIP152; Vincerx Pharma). Enitociclib is a potent and highly selective PTEF-b/CDK9 inhibitor having the structure:

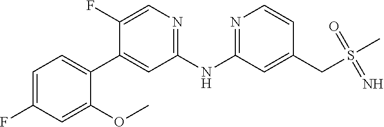 [SMILES: COc1cc(F)ccc1-c1cc(Nc2cc(CS(C)(=N)=O)ccn2)ncc1F]

or a pharmaceutically acceptable salt or hydrate thereof. See, e.g., Anshabo, et al., Frontiers in Oncology, 11 (2021).

In some embodiments, the CDK inhibitor is AZD4573 (AstraZeneca). AZD4573 is a selective inhibitor of CDK9 having the structure:

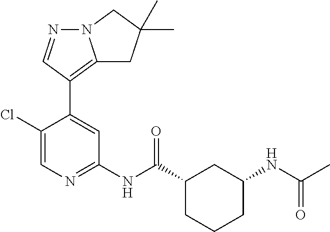 [SMILES: CC(=O)N[C@@H]1CCC[C@H](C(=O)Nc2cc(-c3cnn4c3CC(C)(C)C4)c(Cl)cn2)C1]

or a pharmaceutically acceptable salt or hydrate thereof. See, e.g., Anshabo, et al., Frontiers in Oncology, 11 (2021).

In some embodiments, the CDK inhibitor is i-CDK9. i-CDK9 CDK inhibitor with a 600-fold selectivity for CDK9 over other CDKs, having the structure:

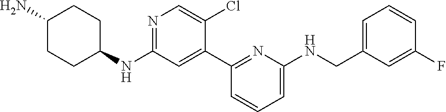 [SMILES: N[C@H]1CC[C@H](Nc2cc(-c3cccc(NCc4cccc(F)c4)n3)c(Cl)cn2)CC1]

or a pharmaceutically acceptable salt or hydrate thereof i-CDK9 inhibits dual-specificity tyrosine-phosphorylation-regulated kinases (DYRK) 1A and 1B, although at a lower potency compared to CDK9. See, e.g., Anshabo, et al., Frontiers in Oncology, 11 (2021).

In some embodiments, the CDK inhibitor is NVP-2. NVP-2 is an ATP-competitive, aminopyrimidine based inhibitor and a chemical analogue of i-CDK9, and has the structure:

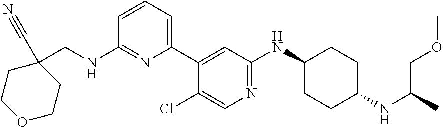 [SMILES: COC[C@@H](C)N[C@H]1CC[C@H](Nc2cc(-c3cccc(NCC4(C#N)CCOCC4)n3)c(Cl)cn2)CC1]

or a pharmaceutically acceptable salt or hydrate thereof. See, e.g., Nat Chem Biol 14, 163-170 (2018).

### Polypeptide Expression and Production

Nucleic acid molecules comprising polynucleotides that encode a DR5-binding polypeptide are provided. In some embodiments, the nucleic acid molecule may also encode a leader sequence that directs secretion of the DR5-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the DR5-binding polypeptide described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a DR5-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lecl3 CHO cells, and FUT8 CHO cells; PER.C6® cells (Crucell); and NSO cells. In some embodiments, the DR5-binding polypeptide may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a DR5-binding polypeptide described herein is provided. The DR5-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a DR5-binding polypeptide that comprises an Fc region. Hydrophobic interactive chromatography, for example, a butyl or phenyl column, may also be suitable for purifying some polypeptides such as antibodies. Ion exchange chromatography (for example anion exchange chromatography and/or cation exchange chromatography) may also be suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

In some embodiments, the DR5-binding polypeptide is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, a DR5-binding polypeptide prepared by the methods described above are provided. In some embodiments, the DR5-binding polypeptide is prepared in a host cell. In some embodiments, the DR5-binding polypeptide is prepared in a cell-free system. In some embodiments, the DR5-binding polypeptide is purified. In some embodiments, a cell culture media comprising a DR5-binding polypeptide is provided.

In some embodiments, compositions comprising antibodies prepared by the methods described above are provided. In some embodiments, the composition comprises a DR5-binding polypeptide prepared in a host cell. In some embodiments, the composition comprises a DR5-binding polypeptide prepared in a cell-free system. In some embodiments, the composition comprises a purified DR5-binding polypeptide.

### Pharmaceutical Compositions

In some embodiments, compositions comprising DR5 agonists, PLK1 inhibitors, and/or CDK inhibitors are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

In some embodiments, INBRX-109 is provided in a formulation comprising 50 mg/mL INBRX-109, 10 mM histidine HCl, 8% w/v sucrose, 0.2% w/v poloxamer-88, pH 6.0.

### Exemplary Methods of Treating Cancer Using a DR5 Agonist and a PLK1 Inhibitor

In some embodiments, methods of treating cancer in an individual comprising administering a DR5 agonist and a PLK1 inhibitor are provided.

In some embodiments, the method comprises administering to the individual an effective amount of a DR5 agonist and a PLK1 inhibitor. Such methods of treatment may be in humans or animals. In some embodiments, methods of treating humans are provided. Nonlimiting exemplary cancers that may be treated with the combination of a DR5 agonists and PLK1 inhibitors provided herein include adrenal cancer; astrocytoma; basal cell carcinoma; biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The DR5 agonist and PLK1 inhibitor can be administered as needed to subjects. Determination of the frequency of administration of each agent can be made by persons skilled in the art, such as an attending physician based on considerations of the condition being treated, age of the subject being treated, severity of the condition being treated, general state of health of the subject being treated and the like. In some embodiments, an effective dose of one or more therapeutic agents is administered to a subject one or more times. In some embodiments, an effective dose of a DR5 agonist and/or a PLK1 inhibitor is administered to the subject daily, semiweekly, weekly, every two weeks, once a month, etc. An effective dose of a DR5 agonist and/or a PLK1 inhibitor is administered to the subject at least once. In some embodiments, the effective dose of a DR5 agonist and/or a PLK1 inhibitor may be administered multiple times, including multiple times over the course of at least a month, at least six months, or at least a year.

In some embodiments, a DR5 agonist is administered in an amount effective for treating (including prophylaxis of) cancer. The therapeutically effective amount is typically dependent on the weight of the subject being treated, his or her physical or health condition, the extensiveness of the condition to be treated, or the age of the subject being treated. In general, DR5-binding polypeptides may be administered in an amount in the range of about 0.05 mg/kg body weight to about 100 mg/kg body weight per dose, or in the range of about 10 g/kg body weight to about 100 mg/kg body weight per dose, or in the range of about 50 g/kg body weight to about 5 mg/kg body weight per dose, or in the range of about 100 g/kg body weight to about 10 mg/kg body weight per dose, or in the range of about 100 g/kg body weight to about 20 mg/kg body weight per dose, or in the range of about 0.5 mg/kg body weight to about 20 mg/kg body weight per dose, or in the range of about 1 mg/kg body weight to about 10 mg/kg body weight per dose.

In some embodiments, a PLK1 inhibitor (or a pharmaceutically acceptable salt thereof or a hydrate) is administered at a dose of 1 mg/m² to 1000 mg/m², including, for example, 10 mg/m² to 500 mg/m², 10 mg/m² to 300 mg/m², or 10 mg/m² to 200 mg/m². In some embodiments, a PLK1 inhibitor (or a pharmaceutically acceptable salt thereof or a hydrate) is administered at a dose of 1 mg to 10,000 mg, including, for example, 10 mg to 5,000 mg, or 10 mg to 1,000 mg, or 10 mg to 500 mg.

In some embodiments, onvansertib is administered at a dose between 2 mg/m2 and 100 mg/m². In some embodiments, volasertib is administered at a dose between 10 and 500 mg. In some embodiments, rigosertib is administered at a dose between 10 and 1,000 mg.

In some embodiments, a therapeutic agent can be administered in vivo by various routes, including, but not limited to, oral, intramuscular, intravenous, intra-arterial, parenteral, intraperitoneal, or subcutaneous. The appropriate formulation and route of administration may be selected according to the intended application.

In some embodiments, a DR5 agonist and a PLK1 inhibitor are administered separately. In some embodiments, a DR5 agonist and a PLK1 inhibitor are administered sequentially. In some embodiments, at least one dose of DR5 agonist is administered before a PLK1 inhibitor. In some embodiments, at least one dose of DR5 agonist is administered after a PLK1 inhibitor.

In some embodiments, a DR5 agonist and a PLK1 inhibitor are administered concurrently.

In some embodiments, a DR5 agonist and a PLK1 inhibitor act synergistically. In some embodiments, synergy is determined in an in vitro cell survival assay. In some embodiments, administration of the DR5 agonist and the PLK1 inhibitor results in a synergistic effect, compared to each agent administered alone.

In some embodiments, a DR5 agonist is provided for use in a method of treating cancer, wherein the method comprises administering the DR5 agonist in combination with a PLK1 inhibitor.

In some embodiments, use of a DR5 agonist for the manufacture of a medicament for treating cancer is provided, wherein the medicament is for administration with a PLK1 inhibitor.

### Exemplary Methods of Treating Cancer Using a DR5 Agonist and a CDK Inhibitor

In some embodiments, methods of treating cancer in an individual comprising administering a DR5 agonist and a CDK inhibitor, such as a CDK9 inhibitor, are provided.

In some embodiments, the method comprises administering to the individual an effective amount of a DR5 agonist and a CDK inhibitor. Such methods of treatment may be in humans or animals. In some embodiments, methods of treating humans are provided. Nonlimiting exemplary cancers that may be treated with the combination of a DR5 agonists and CDK inhibitors provided herein include adrenal cancer; astrocytoma; basal cell carcinoma; biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The DR5 agonist and CDK inhibitor can be administered as needed to subjects. Determination of the frequency of administration of each agent can be made by persons skilled in the art, such as an attending physician based on considerations of the condition being treated, age of the subject being treated, severity of the condition being treated, general state of health of the subject being treated and the like. In some embodiments, an effective dose of one or more therapeutic agents is administered to a subject one or more times. In some embodiments, an effective dose of a DR5 agonist and/or a CDK inhibitor is administered to the subject daily, semiweekly, weekly, every two weeks, once a month, etc. An effective dose of a DR5 agonist and/or a CDK inhibitor is administered to the subject at least once. In some embodiments, the effective dose of a DR5 agonist and/or a CDK inhibitor may be administered multiple times, including multiple times over the course of at least a month, at least six months, or at least a year.

In some embodiments, a DR5 agonist is administered in an amount effective for treating (including prophylaxis of) cancer. The therapeutically effective amount is typically dependent on the weight of the subject being treated, his or her physical or health condition, the extensiveness of the condition to be treated, or the age of the subject being treated. In general, DR5-binding polypeptides may be administered in an amount in the range of about 0.05 mg/kg body weight to about 100 mg/kg body weight per dose, or in the range of about 10 g/kg body weight to about 100 mg/kg body weight per dose, or in the range of about 50 g/kg body weight to about 5 mg/kg body weight per dose, or in the range of about 100 g/kg body weight to about 10 mg/kg body weight per dose, or in the range of about 100 g/kg body weight to about 20 mg/kg body weight per dose, or in the range of about 0.5 mg/kg body weight to about 20 mg/kg body weight per dose, or in the range of about 1 mg/kg body weight to about 10 mg/kg body weight per dose.

In some embodiments, a CDK inhibitor (or a pharmaceutically acceptable salt thereof or a hydrate) is administered at a dose of 1 mg/m² to 1000 mg/m², including, for example, 10 mg/m² to 500 mg/m², 10 mg/m² to 300 mg/m², or 10 mg/m² to 200 mg/m². In some embodiments, a CDK inhibitor (or a pharmaceutically acceptable salt thereof or a hydrate) is administered at a dose of 1 mg to 10,000 mg, including, for example, 10 mg to 5,000 mg, or 10 mg to 1,000 mg, or 10 mg to 500 mg.

In some embodiments, flavopiridol is administered at a dose between 2 mg/m2 and 100 mg/m². In some embodiments, seliciclib is administered at a dose between 10 and 500 mg. In some embodiments, dinaciclib is administered at a dose between 10 and 2,000 mg. In some embodiments, atuveciclib is administered at a dose between 10 and 1,000 mg. In some embodiments, enitociclib is administered at a dose between 10 and 500 mg. In some embodiments, AZD4573 is administered at a dose between 1 and 100 mg.

In some embodiments, a therapeutic agent can be administered in vivo by various routes, including, but not limited to, oral, intramuscular, intravenous, intra-arterial, parenteral, intraperitoneal, or subcutaneous. The appropriate formulation and route of administration may be selected according to the intended application.

In some embodiments, a DR5 agonist and a CDK inhibitor are administered separately. In some embodiments, a DR5 agonist and a CDK inhibitor are administered sequentially. In some embodiments, at least one dose of DR5 agonist is administered before a CDK inhibitor. In some embodiments, at least one dose of DR5 agonist is administered after a CDK inhibitor.

In some embodiments, a DR5 agonist and a CDK inhibitor are administered concurrently.

In some embodiments, a DR5 agonist and a CDK inhibitor act synergistically. In some embodiments, synergy is determined in an in vitro cell survival assay. In some embodiments, administration of the DR5 agonist and the CDK inhibitor results in a synergistic effect, compared to each agent administered alone.

In some embodiments, a DR5 agonist is provided for use in a method of treating cancer, wherein the method comprises administering the DR5 agonist in combination with a CDK inhibitor.

In some embodiments, use of a DR5 agonist for the manufacture of a medicament for treating cancer is provided, wherein the medicament is for administration with a CDK inhibitor.

### Kits

Also provided are articles of manufacture and kits that include any of the DR5 agonists and/or PLK1 inhibitors provided herein and suitable packaging. In some embodiments, the invention includes a kit with (i) a formulation comprising a DR5 agonist, (ii) a formulation comprising a PLK1 inhibitor, and (iii) instructions for using the kit to administer the formulations to an individual. In some embodiments, the invention includes a kit with (i) a formulation comprising a DR5 agonist, and (ii) instructions for using the kit to administer the formulations to an individual in combination with a PLK1 inhibitor. In some embodiments, the invention includes a kit with (i) a formulation comprising a PLK1 inhibitor, and (ii) instructions for using the kit to administer the formulations to an individual in combination with a DR5 agonist.

Also provided are articles of manufacture and kits that include any of the DR5 agonists and/or CDK inhibitors provided herein and suitable packaging. In some embodiments, the invention includes a kit with (i) a formulation comprising a DR5 agonist, (ii) a formulation comprising a CDK inhibitor, and (iii) instructions for using the kit to administer the formulations to an individual. In some embodiments, the invention includes a kit with (i) a formulation comprising a DR5 agonist, and (ii) instructions for using the kit to administer the formulations to an individual in combination with a CDK inhibitor. In some embodiments, the invention includes a kit with (i) a formulation comprising a CDK inhibitor, and (ii) instructions for using the kit to administer the formulations to an individual in combination with a DR5 agonist.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the DR5 agonists PLK1 inhibitors, and/or CDK inhibitors generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous solution of DR5 agonist.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Combinatorial Activity of INBRX-109 and Onvansertib

The combination of INBRX-109 and onvansertib was tested on various cancer cell lines to determine cytotoxicity to cancer cells.

### Assay Protocol

On day 1, cells were seeded as follows. Monolayer cultures of each cell line were harvested for compound screening as detailed below. Culture medium was aspirated, and the cells were washed once with PBS. Accutase was added and flasks were incubated at 37° C. until cells became detached. An equal volume of complete medium was added to quench the Accutase, and the cells were then pipetted up and down several times to generate a homogenous single cell suspension. The density and viability of cells was determined by Trypan Blue using a TC20 Automated Cell Counter. Experimental cells were resuspended to a concentration of 0.17×10\hat{ }6/mL in Eagle's Minimum Essential Medium (EMEM)/10% FBS/Anti-Anti media (complete EMEM) and seeded in the inner wells of 384-well luminescence plates at 15 μL/well (2,500/well final). Each cell line was plated in duplicate on separate plates. Outer wells were filled with 50 μL PBS, then plates were incubated in a humidified temperature-controlled 37° C. tissue culture incubator at 5% CO₂ overnight for 16 hours.

On day 2, the following test and control articles were prepared: onvansertib, INBRX-109, and Staurosporine.

Onvansertib: A 10 mg stock of onvansertib was purchased from Selleck Chemicals and was resuspended to 10 mM in DMSO. This was then aliquoted and stored at −80° C. Aliquots were thawed immediately prior to dilution and use in the assay. A 500× master plate of serial dilutions (6-point 5-fold dilutions in 100% DMSO starting at 500 μM, plus a DMSO only control) was prepared and mixed gently with a pipette. To generate a 5× working dilution plate of onvansertib, a 1:100 dilution was performed from the 500× plate into complete media (EMEM).

INBRX-109: INBRX-109 assay concentration ranges were chosen to bracket the minimal and maximal activity seen in previous cytotoxicity assays with several cancerous cell lines, with 1 nM defined as the maximally efficacious concentration. A 50× master plate of INBRX-109 serial dilutions (6-point 10-fold dilutions in complete EMEM starting at 500 nM, plus a complete EMEM only control) was prepared and mixed gently with pipette. To generate a 5× working dilution plate of INBRX-109, each well from the 50× master plate was diluted 1:10 into complete EMEM.

Staurosporine: Staurosporine was included in the assay as a positive control for cytotoxicity. A 10 mM DMSO stock purchased from the manufacturer was thawed, aliquoted, and stored at −80° C. Aliquots were thawed immediately prior to dilution and use in the assay. A 5× Staurosporine working dilution (100 μM) was made by adding 5 μL of a 10 mM Staurosporine stock solution to 495 μL complete EMEM, then mixed well.

Also on day 2, test and control articles were added. Onvansertib small molecule working dilutions (5 μL of 5×), INBRX-109 working dilutions (5 μL of 5×), or Staurosporine positive control (5 μL of 5×) were added to the respective experimental wells. Onvansertib titrations were performed going across the plate, and INBRX-109 titrations were done going down the plate, leading to a matrix of all possible combinations of the two test articles. These test articles were added, in duplicate, to each cell line. The plates were then centrifuged at 400× g for 1 minute, then incubated in a 37° C. humidity-controlled tissue culture incubator (5% CO₂) for 48 hours.

On day 4, viability measurements were taken. Plates were equilibrated to room temperature for 10 minutes, then 25 μL of CellTiter-Glo 2.0® was added to each well. Plates were spun for 1 minute at 400×g, then covered and incubated in the dark for 10 minutes at room temperature. Any visible bubbles were removed with 100% ethanol vapor, then luminescence (RLU) was read on the Spectra Max M5e plate reader, using the 384-well opaque plate setting and SoftMaxPro v5.4 software with an integration time of 50 milliseconds. To determine the effect of test articles on cell viability, raw RLU values were exported to Excel and percent survival was calculated as a percentage of vehicle control (0.5% DMSO in EMEM), where the vehicle control is set at 100%. Data was graphed in GraphPad Prism 9.

### Results

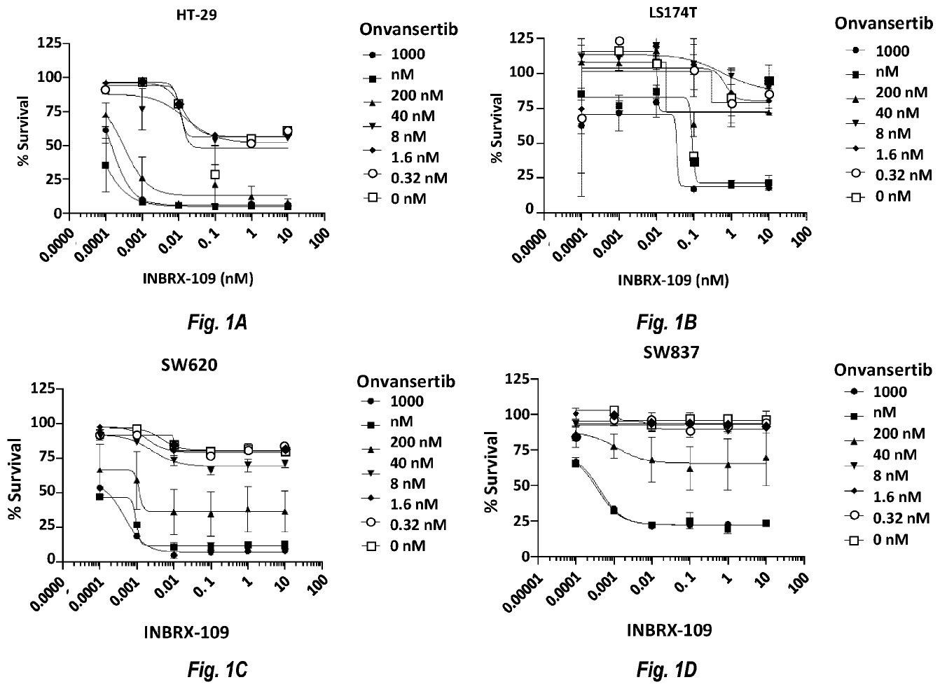

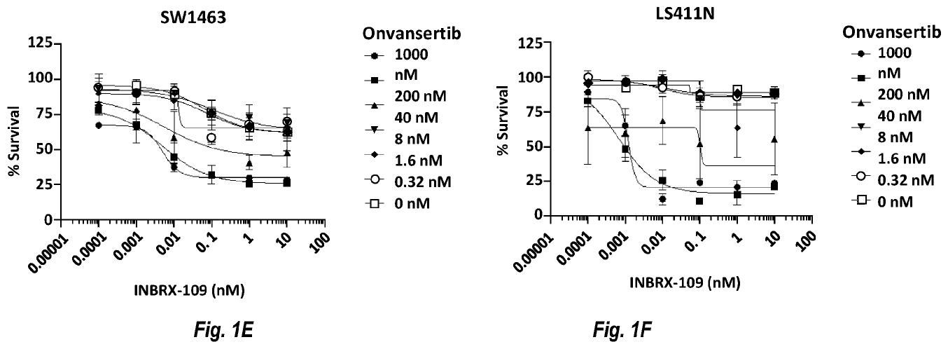

FIG. 1A-1F shows results of a titration experiment in which various cancer cell lines were contacted with different concentrations of INBRX-109 (0.0001, 0.001, 0.01, 0.1, 1, or 10 nM) and onvansertib (0, 0.32, 1.6, 8, 40, 200, or 1000 nM). Percent survival of cancer cells is shown on the y-axis of each graph. Results are shown for cancer cell lines HT-29, LS174T, SW620, SW837, SW1463, and LS411N.

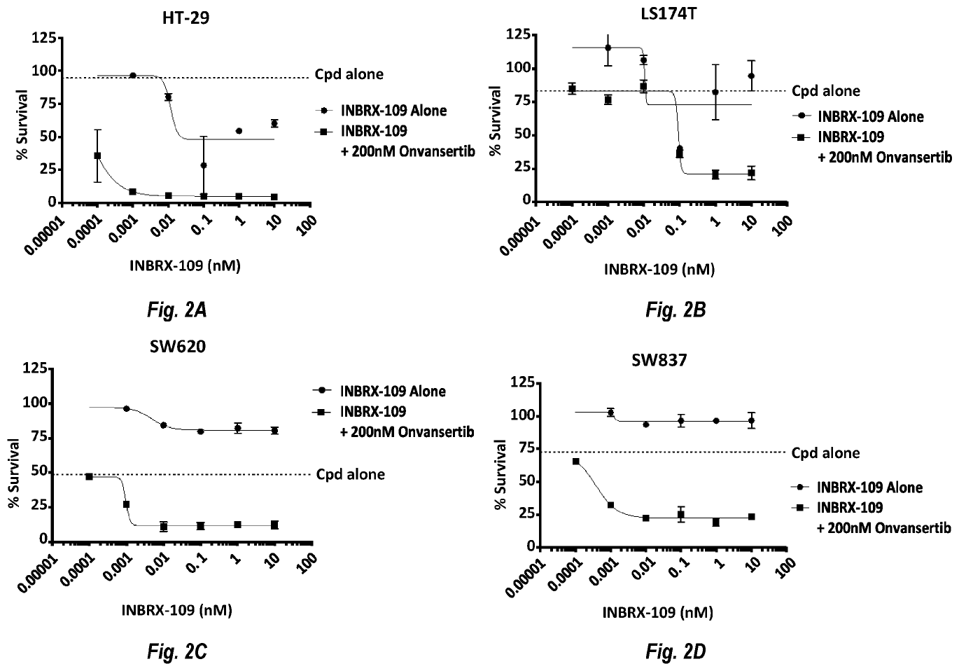

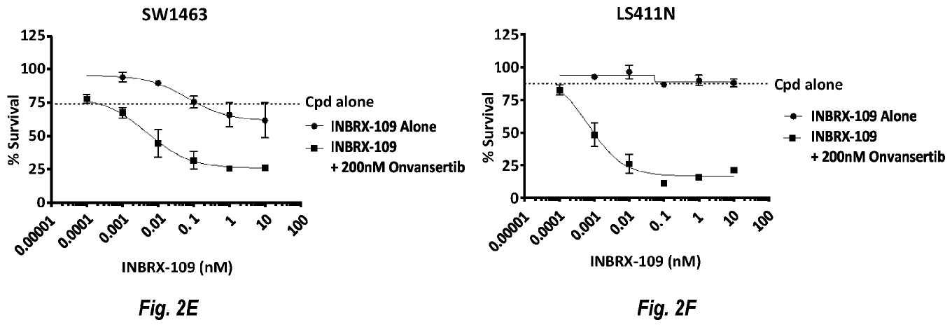

FIG. 2A-2F shows results of an INBRX-109 titration experiment in which various cancer cell lines were contacted with different concentrations of INBRX-109 (0.0001, 0.001, 0.01, 0.1, 1, or 10 nM), alone or in combination with 200 nM onvansertib. Percent survival of cancer cells is shown on the y-axis of each graph. Results are shown for cancer cell lines HT-29, LS174T, SW620, SW837, SW1463, and LS411N. The dotted line labeled “Cpd alone” shows the percent survival of cancer cells treated with 200 nM onvansertib alone.

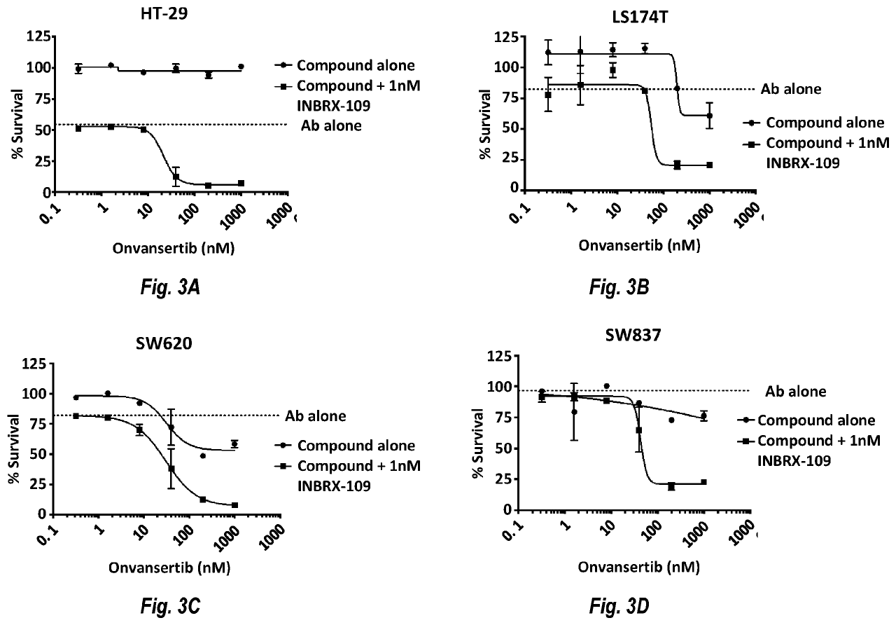

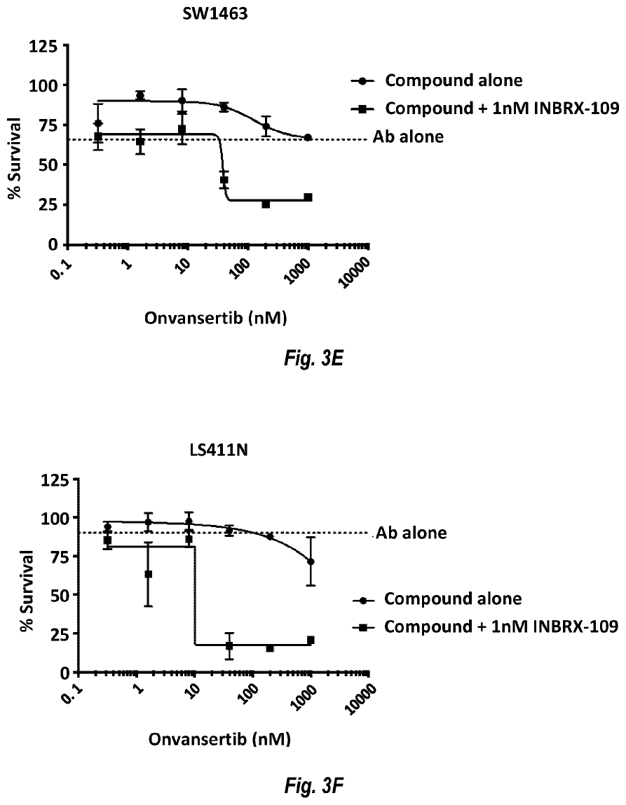

FIG. 3A-3F shows results of an onvansertib titration experiment in which various cancer cell lines were contacted with different concentrations of onvansertib (0.32, 1.6, 8, 40, 200, or 1000 nM), alone or in combination with 1 nM INBRX-109. Percent survival of cancer cells is shown on the y-axis of each graph. Results are shown for cancer cell lines HT-29, LS174T, SW620, SW837, SW1463, and LS411N. The dotted line labeled “Ab alone” shows the percent survival of cancer cells treated with INBRX-109 alone.

Table 1 shows the EC₅₀ values of the INBRX-109 titration curves with and without 200 nM onvansertib. Maximum cytotoxicity was calculated by subtracting the % survival from 100.

TABLE 1

INBRX-109 + 200 nM
Onvansertib

| | |
|---|---|
| INBRX-109 Only | Max |

| | EC₅₀ | Max | EC₅₀ | Cytotox |
|---|---|---|---|---|
| Cell Line | (pM) | Cytotox % | (pM) | (%) |
| HT-29 | 11.58 | 71.42 | 0.0554 | 94.94 |
| LS174T | 10.65 | 59.68 | 90.66 | 79.32 |
| SW620 | 4.775 | 20.25 | 0.965 | 88.56 |
| SW837 | 1.234 | 6.43 | 0.371 | 80.64 |
| SW1463 | 65.98 | 38.14 | 4.946 | 73.74 |
| LS411N | Unstable | 13.27 | 0.687 | 88.9 |

Table 2 shows the EC₅₀ values of the onvansertib titration curve with and without 1 nM INBRX-109. Maximum cytotoxicity was calculated by subtracting the % survival from 100.

TABLE 2

| | Onvansertib Only | | Onvansertib + 1 nM INBRX-109 | |
|---|---|---|---|---|
| | EC₅₀ | Max Cytotox | EC₅₀ | Max Cytotox |
| Cell Line | (nM) | at 200 nM (%) | (nM) | at 200 nM (%) |
| HT-29 | ~2.296 | 5.54 | 21.41 | 94.63 |
| LS174T | ~196.7 | 16.93 | ~54.73 | 79.32 |
| SW620 | 31.3 | 51.37 | 30.45 | 87.53 |
| SW837 | 27247 | 27.17 | ~43.16 | 80.64 |
| SW1463 | 124.3 | 25.79 | ~38.19 | 74.39 |
| LS411N | ~4145862 | 12.35 | ~10.27 | 84.45 |

The combination of INBRX-109 and onvansertib showed increased cell killing on every cancer cell line tested in this screen. Greater benefits were generally observed at onvansertib concentrations higher than 40 nM. At these concentrations, the combination yielded a synergistic effect, as was apparent by the shift in the EC₅ of INBRX-109, compared to the EC₅₀ of INBRX-109 treatment alone. See, e.g., FIG. 2 and Table 1. Additionally, this combination led to a decrease in overall cell survival compared to treatment with either drug alone.

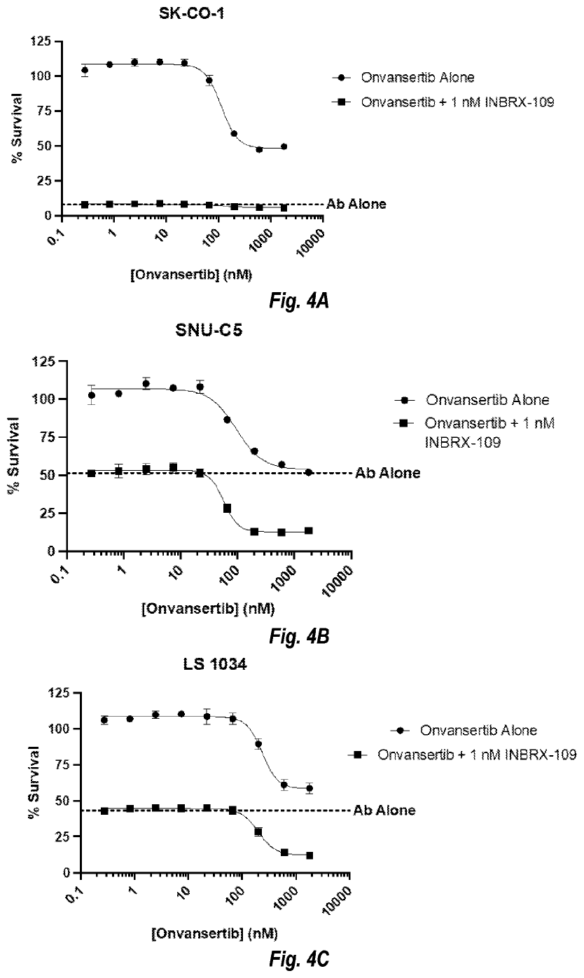

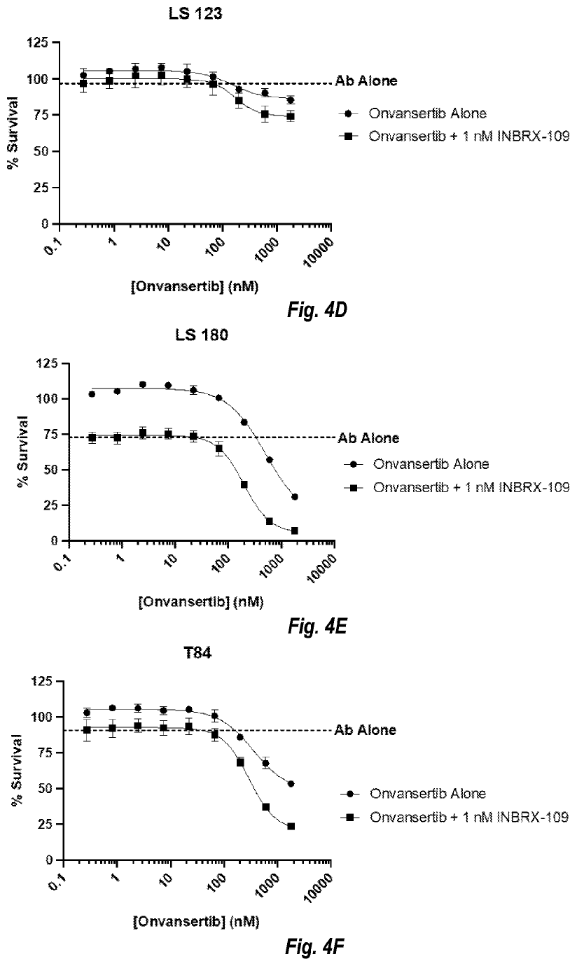

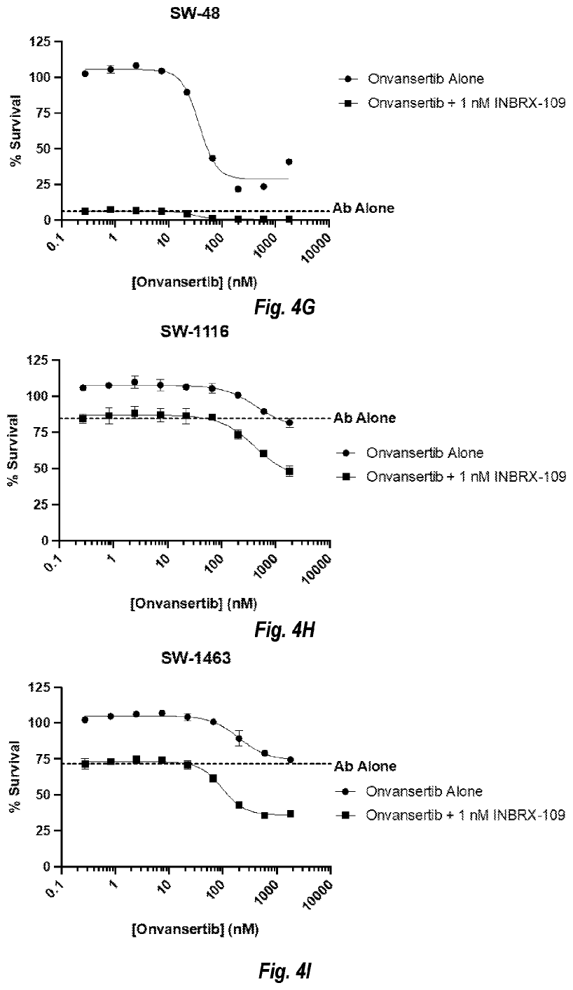

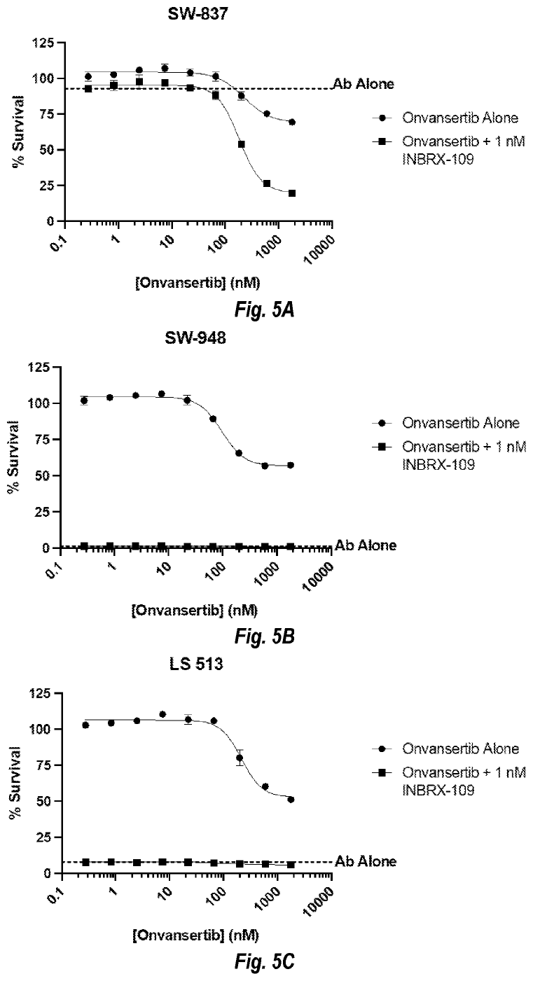

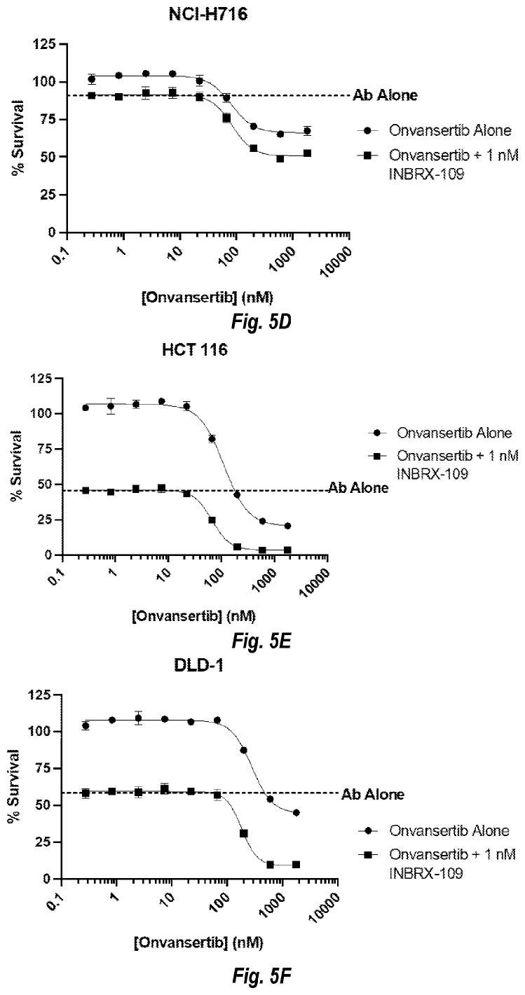

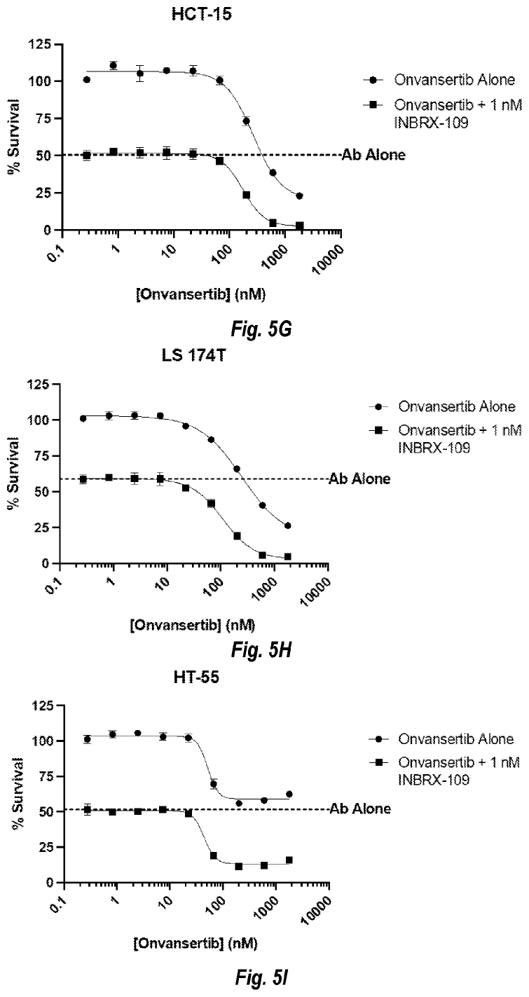

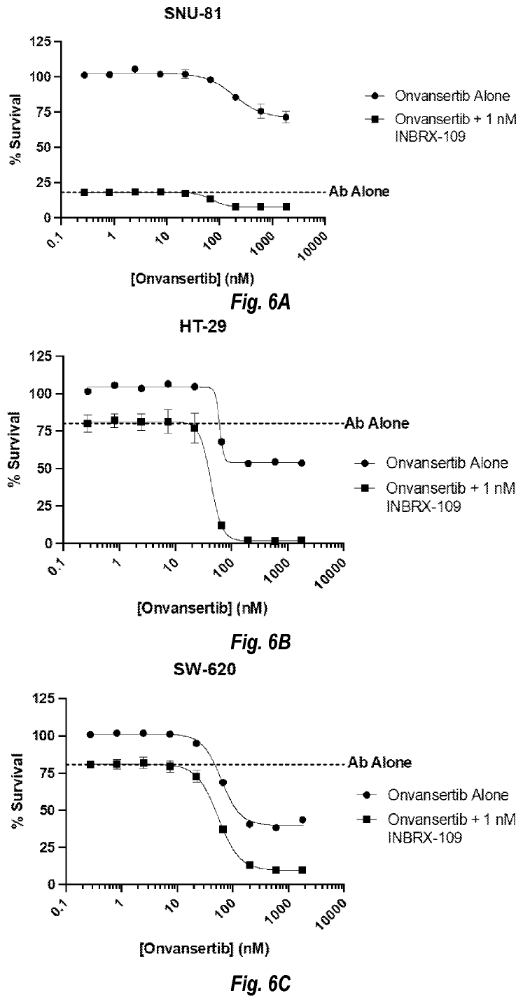

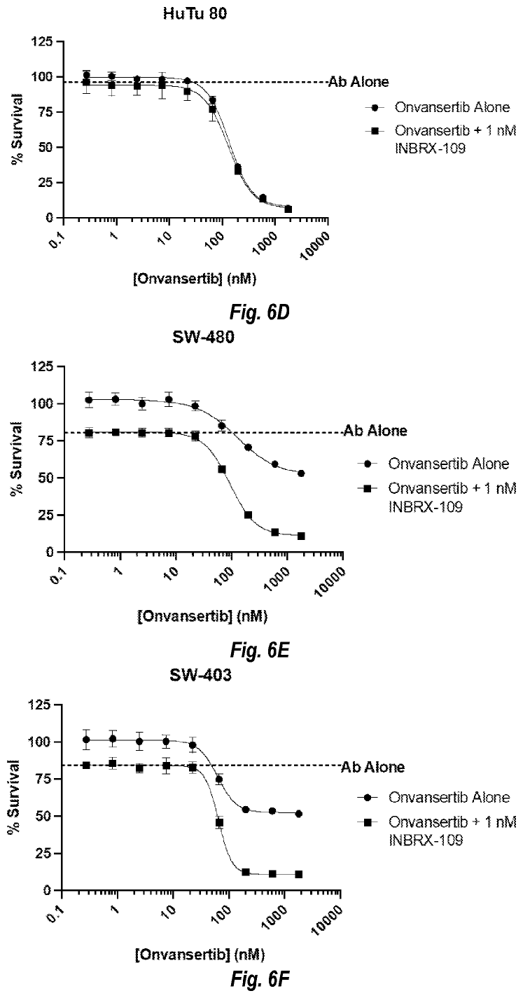

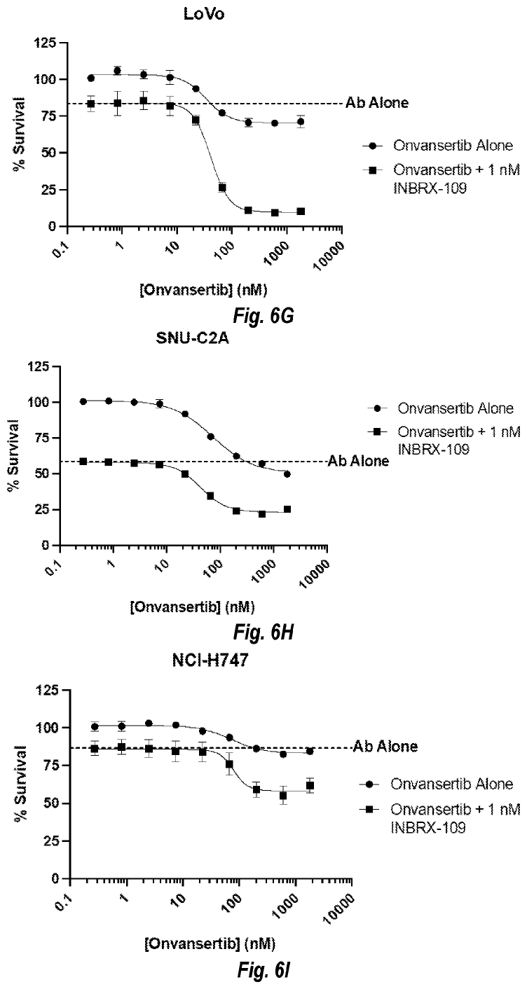

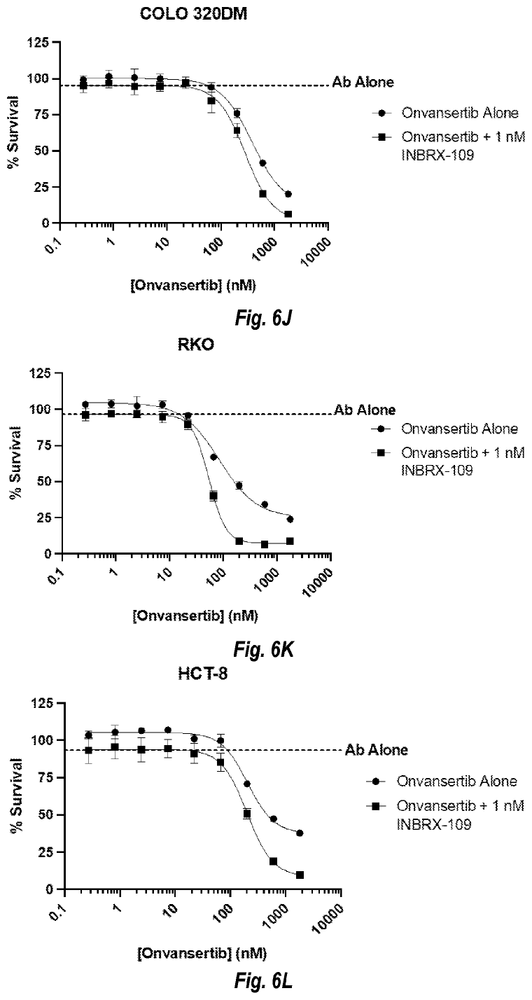

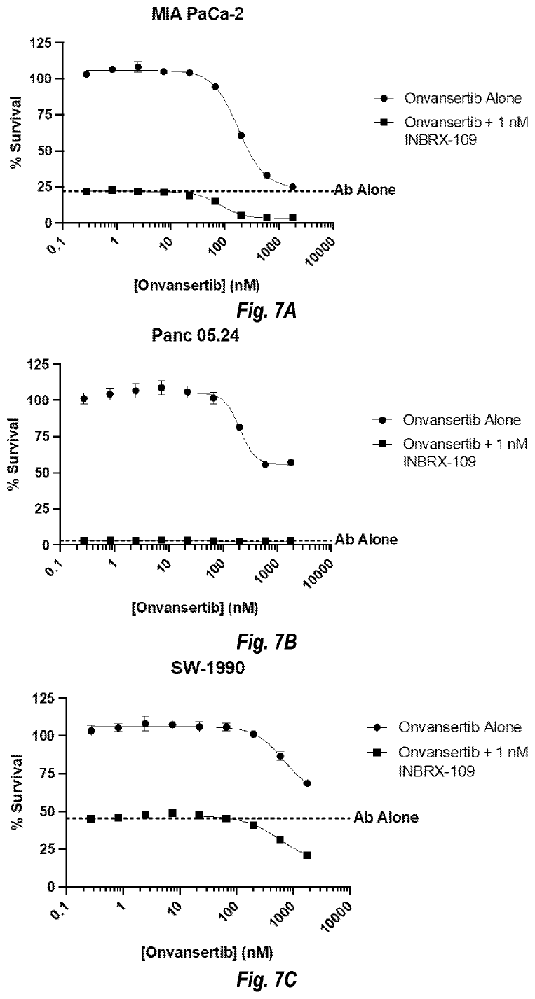

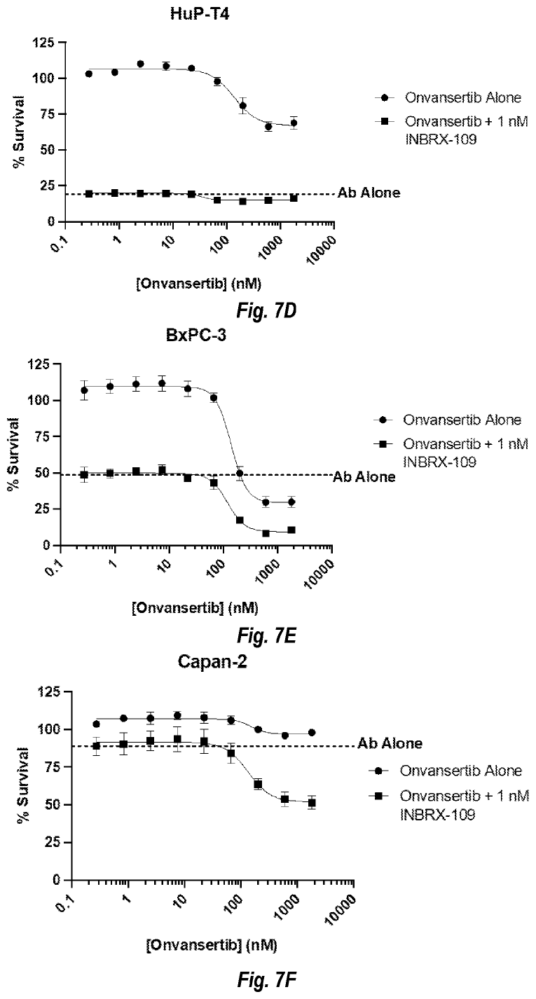

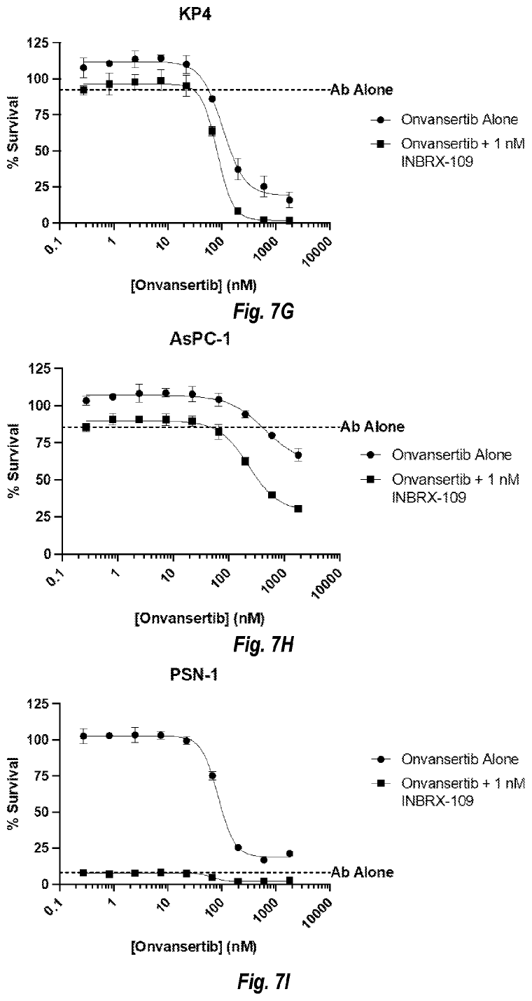

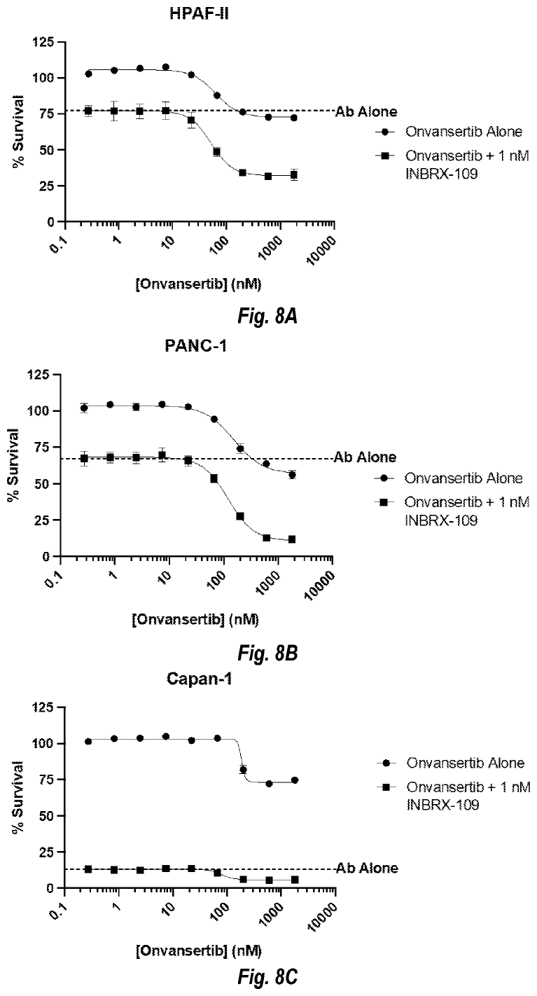

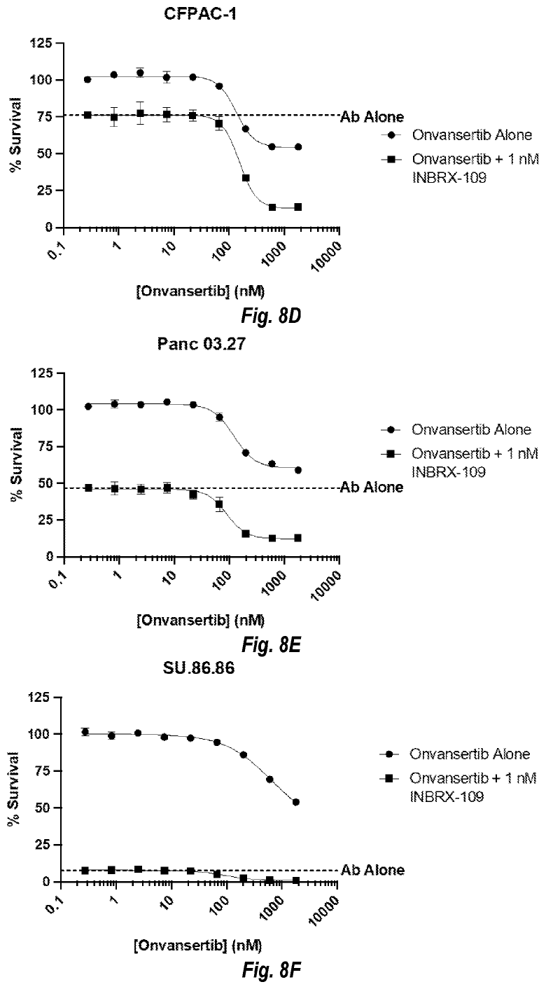

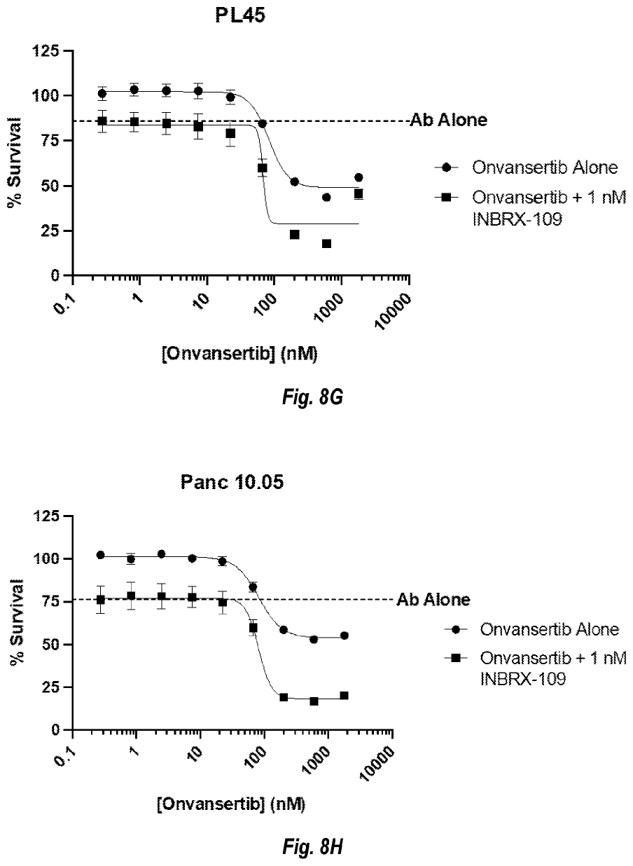

Additional cancer cell lines were assayed for cell killing in the presence of 1 nM INBRX-109 alone, onvansertib alone, or the combination of 1 nM INBRX-109 and different concentrations of onvansertib (274 pM, 823 pM, 2.47 nM, 7.41 nM, 22.22 nM, 66.67 nM, 200 nM, 600 nM, and 1.8 M), substantially as described above. Tables 3 and 4 and FIGS. 4-8 show the maximum cytotoxicity of 1 nM INBRX-109 alone, and EC₅₀ values of the onvansertib titration curve with and without 1 nM INBRX-109. The results for a panel of colorectal cancer cell lines are shown in Table 3 and FIGS. 4-6, and the results for a panel of pancreatic cell lines are shown in Table 4 and FIGS. 7 and 8.

TABLE 3

| | 1 nM INBRX- | | | | Onvansertib + 1 nM | |
|---|---|---|---|---|---|---|
| | 109 Only | | Onvansertib Only | | INBRX-109 | |
| | | Max | | Max | | Max |
| | | Cytotox at | | Cytotox at | | Cytotox at |
| Cell Line | EC₅₀ (nM) | 1 nM (%) | EC₅₀ (nM) | 600 nM (%) | EC₅₀ (nM) | 600 nM (%) |
| SK-CO-1 | N/A | 92.2 | 113.4 | 52.8 | 111.0 | 94.2 |
| SNU-C5 | N/A | 48.6 | 95.6 | 42.9 | 57.7 | 87.7 |
| LS 1034 | N/A | 56.9 | 236.6 | 38.8 | 199.4 | 85.8 |
| LS 123 | N/A | 3.1 | 150.1 | 9.6 | 171.6 | 24.1 |
| LS 180 | N/A | 27.2 | 505.0 | 42.9 | 196.6 | 86.2 |
| T84 | N/A | 9.1 | 355.3 | 32.1 | 297.2 | 62.9 |
| SW-48 | N/A | 93.9 | 37.3 | 76.6 | 28.3 | 99.4 |
| SW-1116 | N/A | 15.3 | 441.4 | 10.4 | 411.1 | 39.4 |
| SW-1463 | N/A | 28.5 | 201.1 | 21.0 | 98.0 | 64.2 |
| SW-837 | N/A | 7.1 | 226.7 | 24.5 | 183.5 | 73.5 |
| SW-948 | N/A | 98.5 | 97.0 | 43.2 | 23.3 | 99.0 |
| LS 513 | N/A | 92.3 | 215.0 | 39.6 | 100.9 | 93.7 |
| NCI-H716 | N/A | 9.2 | 81.3 | 34.8 | 83.6 | 51.2 |
| HCT 116 | N/A | 54.4 | 112.0 | 76.0 | 66.8 | 96.6 |
| DLD-1 | N/A | 41.9 | 280.0 | 45.8 | 183.6 | 90.4 |
| HCT-15 | N/A | 49.6 | 268.0 | 61.4 | 175.3 | 94.9 |
| LS174T | N/A | 41.2 | 251.7 | 59.5 | 109.1 | 94.1 |
| HT-55 | N/A | 48.4 | 51.4 | 41.8 | 44.1 | 87.9 |
| SNU-81 | N/A | 82.1 | 187.3 | 24.3 | 71.9 | 92.3 |
| HT-29 | N/A | 19.9 | 61.6 | 45.6 | 43.0 | 98.3 |
| SW620 | N/A | 19.4 | 62.1 | 61.8 | 54.6 | 90.4 |
| Hutu 80 | N/A | 4.0 | 137.8 | 85.4 | 133.4 | 86.5 |
| SW-480 | N/A | 19.7 | 126.0 | 40.7 | 93.3 | 86.3 |
| SW-403 | N/A | 15.7 | 62.5 | 46.4 | 64.9 | 89.0 |
| LoVo | N/A | 16.2 | 34.1 | 29.6 | 41.8 | 90.8 |
| SNU-C2A | N/A | 41.4 | 72.7 | 42.9 | 42.9 | 78.0 |
| NCI-H747 | N/A | 13.4 | 71.6 | 17.4 | 78.7 | 44.5 |
| COLO320DM | N/A | 4.7 | 382.5 | 58.3 | 283.0 | 79.6 |
| RKO | N/A | 3.6 | 85.9 | 65.7 | 54.7 | 93.8 |
| HCT-8 | N/A | 6.7 | 205.0 | 52.7 | 200.0 | 81.3 |

TABLE 4

| | 1 nM | | | | Onvansertib + | |
|---|---|---|---|---|---|---|
| | INBRX- | | Onvansertib | | 1 nM | |
| | 109 Only | | Only | | INBRX-109 | |
| | | Max | | Max | | Max |
| | | Cytotox | | Cytotox at | | Cytotox |
| | EC₅₀ | at 1 | EC₅₀ | 600 nM | EC₅₀ | at 600 |
| Cell Line | (nM) | nM (%) | (nM) | (%) | (nM) | nM (%) |
| MIA PaCa-2 | N/A | 77.9 | 179.0 | 67.0 | 81.3 | 96.4 |
| Panc 05.04 | N/A | 97.0 | 204.9 | 44.4 | 61.1 | 97.4 |
| SW-1990 | N/A | 54.7 | 699.7 | 13.4 | 598.4 | 68.7 |
| HuP-T4 | N/A | 80.7 | 138.1 | 33.7 | 35.1 | 85.3 |
| BxPC-3 | N/A | 51.3 | 139.4 | 70.1 | 119.3 | 91.8 |
| Capan-2 | N/A | 11.1 | 149.7 | 3.9 | 134.5 | 46.2 |
| KP4 | N/A | 7.8 | 104.7 | 74.7 | 82.4 | 97.8 |
| AsPC-1 | N/A | 14.5 | 444.3 | 19.9 | 230.3 | 60.0 |
| PSN-1 | N/A | 91.9 | 85.3 | 83.2 | 63.0 | 98.0 |
| HPAF-II | N/A | 22.8 | 63.3 | 27.2 | 51.9 | 68.2 |
| PANC-1 | N/A | 32.6 | 152.1 | 36.2 | 120.8 | 87.2 |
| Capan-1 | N/A | 86.9 | 186.2 | 27.8 | 79.0 | 94.3 |
| CFPAC-1 | N/A | 23.8 | 134.5 | 45.3 | 153.7 | 86.4 |
| Panc 03.27 | N/A | 53.2 | 121.5 | 36.7 | 90.0 | 87.5 |
| SU.86.86 | N/A | 92.5 | 629.1 | 30.5 | 91.4 | 98.9 |
| PL45 | N/A | 14.1 | 83.6 | 56.5 | 68.1 | 82.2 |
| Panc 10.05 | N/A | 23.8 | 81.9 | 47.2 | 81.7 | 83.3 |

The combination of INBRX-109 and onvansertib showed increased cell killing on the cancer cell line tested in this screen, compared to either agent alone, and in some cases, the addition of 1 nM INBRX-109 substantially reduced the EC50, compared to onvansertib alone.

This data suggests that the combination of a DR5 agonist, such as INBRX-109, and a PLK1 inhibitor, such as onvansertib, results in improved or synergistic cancer cell killing compared the individual drugs themselves.

### Example 2: Combinatorial Activity of INBRX-109 and CDK9 Inhibitors

Additional assays were performed to test the combination of INBRX-109 and a cyclin-dependent kinase 9 (CDK9) inhibitor (dinaciclib, NVP-2, flavopiridol, and enitociclib), or a downstream MCL-1 inhibitor (AZD5991) on various cell lines to determine cytotoxicity to cancer cells.

### Assay Protocol

On day 1, cells were seeded as follows. Monolayer cultures of each cell line (chondrosarcoma: CAL-78, OUMS-27, SW1353, and H-EMC-SS; colorectal cancer: SW620; and synovial sarcoma: HS-SY-II) were harvested for compound screening. Culture medium was aspirated, and the cells were washed once with PBS. Accutase was added and flasks were incubated at 37° C. until cells became detached. An equal volume of complete medium was added to quench the Accutase, and the cells were then pipetted up and down several times to generate a homogenous single cell suspension. The density and viability of cells was determined by Trypan Blue using a TC20 Automated Cell Counter. Experimental cells were resuspended to a concentration of 0.17×10\hat{ }6/mL in Eagle's Minimum Essential Medium (EMEM)/10% FBS/Anti-Anti media (complete EMEM) and seeded in the inner wells of 384-well luminescence plates at 15 mL/well (2,500/well final). Each cell line was plated in duplicate on separate plates. Outer wells were filled with 50 mL PBS, then plates were incubated in a humidified temperature-controlled 37° C. tissue culture incubator at 5% CO2 overnight for 16 hours.

On day 2, the following test articles and INBRX-109 were prepared as provided below:
Dinaciclib (purchased from MedChemExpress): A 5× working serial dilution plate was prepared (6-point, 5-fold dilutions starting at 312.5 uM for a final concentration range of 625 nM to 0.2 nM) in complete media (EMEM).
NVP-2 (purchased from MedChemExpress): A 5× working serial dilution plate was prepared (6-point, 5-fold dilutions starting at 312.5 uM for a final concentration range of 625 nM to 0.2 nM) in complete media (EMEM).
Flavopiridol (purchased from MedChemExpress): A 5× working serial dilution plate was prepared (6-point, 5-fold dilutions starting at 312.5 uM for a final concentration range of 625 nM to 0.2 nM) in complete media (EMEM).
Enitociclib (purchased from MedChemExpress): A 5× working serial dilution plate was prepared (6-point, 5-fold dilutions starting at 312.5 uM for a final concentration range of 625 nM to 0.2 nM) in complete media (EMEM).
AZD-5991 (purchased from MedChemExpress): A 5× working serial dilution plate was prepared (6-point, 5-fold dilutions starting at 5 mM for a final concentration range of 10 uM to 3.2 nM) in complete media (EMEM).
INBRX-109: A 5× working serial dilution plate (6-point, 10-fold dilutions starting at 50 nM for a final concentration range of 10 nM to 0.0001 nM) in complete media (EMEM).

The test articles were added to the cells as follows: Small molecule compound working dilutions (5 μL of 5×), INBRX-109 working dilutions (5 μL of 5×), or media only were added to the respective experimental wells. Small molecule compound titrations were performed going across the plate, and INBRX-109 titrations were done going down the plate, leading to a matrix of all possible combinations of the two test articles. These test articles were added, in duplicate, to each cell line. The plates were then centrifuged at 400×g for 1 minute, then incubated in a 37° C. humidity-controlled tissue culture incubator (5% CO₂) for 48 hours.

After incubation with the test articles, viability measurements were taken, briefly. Plates were equilibrated to room temperature for 10 minutes, then 25 μL of CellTiter-Glo 2.0® was added to each well. Plates were spun for 1 minute at 400×g, then covered and incubated in the dark for 10 minutes at room temperature. Any visible bubbles were removed with 100% ethanol vapor, then luminescence (RLU) was read on the Spectra Max M5e plate reader, using the 384-well opaque plate setting and SoftMax® Pro v5.4 software with an integration time of 50 milliseconds. To determine the effect of test articles on cell viability, raw RLU values were exported to Excel and percent survival was calculated as a percentage of vehicle control (0.5% DMSO in EMEM), where the vehicle control is set at 100%. Data was graphed in GraphPad Prism 9.

### Results

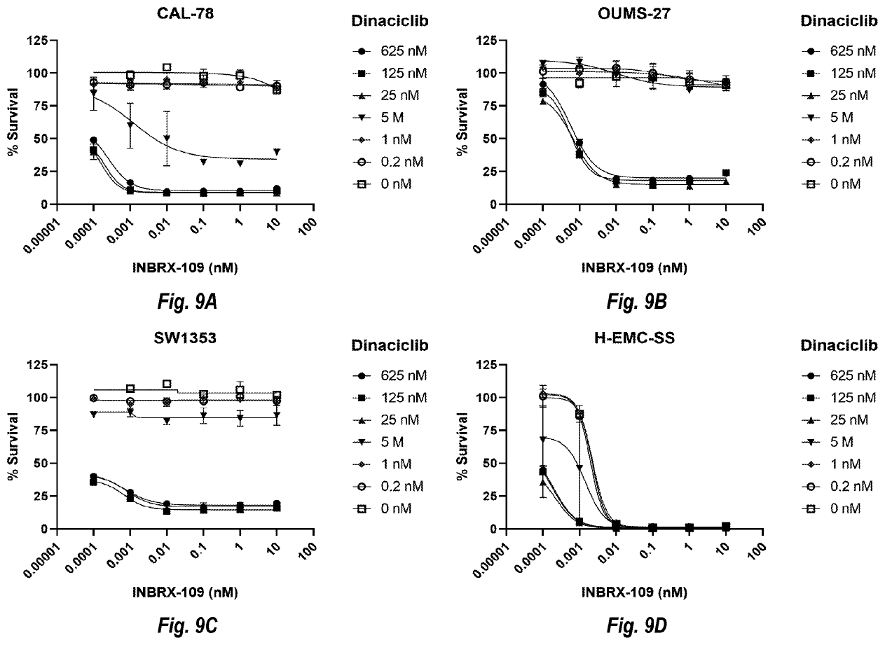

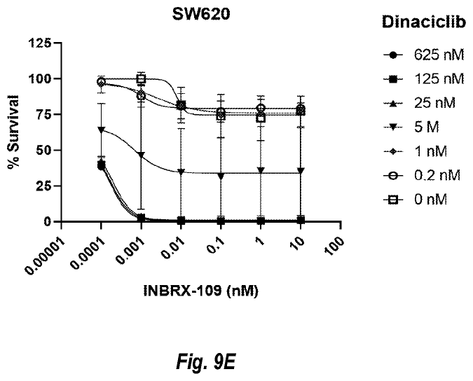

The results of these titration experiments are shown in FIGS. 9A-9E (INBRX-109 and dinaciclib titrated), 10A-10E (INBRX-109 titrated alone or with 25 nM dinaciclib), 11A-11E (dinaciclib titrated alone or with 1 nM INBRX-109), 12A-12E (INBRX-109 and NVP-2 titrated), 13A-13E (INBRX-109 titrated alone or with 125 nM NVP-2), 14A-14E (NVP-2 titrated alone or with 1 nM INBRX-109), 15A-15F (INBRX-109 and flavpirdol titrated), 16A-16F (INBRX-109 titrated alone or with 125 nM flavopiridol), 17A-17F (flavopiridol titrated alone or with 1 nM INBRX-109), 18A-18F (INBRX-109 and enitociclib titrated), 19A-19F (INBRX-109 titrated alone or with 125 nM enitociclib), 20A-20F (enitociclib titrated alone or with 1 nM INBRX-109), 21A-21F (INBRX-109 and AZD-5991 titrated), 22A-22F (INBRX-109 titrated alone or with 2 nM AZD-5991), and 23A-23F (AZD-5991 titrated alone or with 1 nM INBRX-109). The dotted line labeled “Ab alone” shows the percent survival of cancer cells treated with 1 nM INBRX-109 alone and the dotted line labeled “Cpd alone” shows the percent survival of cancer cells treated with the indicated small molecule inhibitor.

Table 5 shows the EC50 values of the INBRX-109 titration curves with and without 25 nM dinaciclib. Maximum cytotoxicity was calculated by subtracting the % survival from 100.

TABLE 5

| | INBRX-109 Only | | INBRX-109 + 25 nM | |
|---|---|---|---|---|
| | | Max | Dinaciclib | |
| | EC₅₀ | Cytotox (%) | EC₅₀ | Max |
| Cell Line | (pM) | at 1 nM | (pM) | Cytotox % |
| CAL-78 | >3597 | 9.3 | 0.16 | 91.7 |
| OUMS-27 | 982.2 | 6.4 | 0.68 | 86.1 |
| SW1353 | unstable | 6.9 | 0.81 | 84.6 |
| H-EMC-SS | 2.2 | 99.1 | 0.22 | 99.7 |
| SW620 | 7.8 | 27.6 | 0.20 | 99.1 |

Table 6 shows the EC50 values of the dinaciclib titration curve with and without 1 nM INBRX-109. Maximum cytotoxicity was calculated by subtracting the % survival from 100.

TABLE 6

| | Dinaciclib Only | | Dinaciclib + 1 nM | |
|---|---|---|---|---|
| | | Max | INBRX-109 | |
| | EC₅₀ | Cytotox at | EC₅₀ | Max |
| Cell Line | (nM) | 25 nM (%) | (nM) | Cytotox (%) |
| CAL-78 | 5.76 | 47.1 | 3.70 | 91.4 |
| OUMS-27 | Unstable | 15.1 | 6.18 | 86.13 |
| SW1353 | 8.23 | 58.0 | 7.38 | 85.11 |
| H-EMC-SS | 4.62 | 54.0 | 7.61 | 99.62 |
| SW620 | 3.82 | 46.2 | 4.80 | 99.52 |

Table 7 shows the EC50 values of the INBRX-109 titration curves with and without 25 nM NVP-2. Maximum cytotoxicity was calculated by subtracting the % survival from 100.

TABLE 7

| | INBRX-109 Only | | INBRX-109 + 125 nM NVP-2 | |
|---|---|---|---|---|
| | | Max | | Max |
| | EC₅₀ | Cytotox (%) | EC₅₀ | Cytotox |
| Cell Line | (pM) | at 1 nM | (pM) | (%) |
| CAL-78 | unstable | 9.3 | 0.13 | 82.2 |
| OUMS-27 | >4090 | 4.2 | 0.42 | 84.3 |
| SW1353 | unstable | 0.8 | 0.50 | 71 |
| H-EMC-SS | 1.96 | 96.5 | 0.17 | 98.8 |
| SW620 | 98.06 | 20.8 | 0.11 | 98.1 |

Table 8 shows the EC50 values of the NVP-2 titration curve with and without 1 nM INBRX-109. Maximum cytotoxicity was calculated by subtracting the % survival from 100.

TABLE 8

| | NVP-2 Only | | NVP-2 + 1 nM INBRX-109 | |
|---|---|---|---|---|
| | | Max | | Max |
| | EC₅₀ | Cytotox at | EC₅₀ | Cytotox |
| Cell Line | (nM) | 125 nM (%) | (nM) | (%) |
| CAL-78 | 23.25 | 42.2 | 26.44 | 92.4 |
| OUMS-27 | 9.0 | 20.4 | 16.31 | 88.5 |
| SW1353 | 30.63 | 52.8 | 22.28 | 79.5 |
| H-EMC-SS | 23.51 | 46.4 | 24.46 | 98.7 |
| SW620 | 12.57 | 39.5 | 4.91 | 98.7 |

Table 9 shows the EC50 values of the INBRX-109 titration curves with and without 25 nM flavopiridol. Maximum cytotoxicity was calculated by subtracting the % survival from 100.

TABLE 9

| | INBRX-109 Only | | INBRX-109 + 125 nM | |
|---|---|---|---|---|
| | | Max | Flavopiridol | |
| | EC₅₀ | Cytotox (%) | EC₅₀ | Max |
| Cell Line | (pM) | at 1 nM | (pM) | Cytotox (%) |
| CAL-78 | 17.19 | 11 | 0.60 | 77.3 |
| OUMS-27 | 0.95 | 6.2 | 0.89 | 61.3 |
| HS-SY-II | Unstable | 8.5 | 1.93 | 64.1 |
| SW1353 | Unstable | 4.1 | 2.81 | 39.8 |
| H-EMC-SS | 9.25 | 96.2 | 1.46 | 98.5 |
| SW620 | 1.37 | 8.2 | 0.90 | 90.8 |

Table 10 shows the EC50 values of the flavopiridol titration curve with and without 1 nM INBRX-109. Maximum cytotoxicity was calculated by subtracting the % survival from 100.

TABLE 10

| | Flavopiridol Only | | Flavopiridol + 1 nM | |
|---|---|---|---|---|
| | | Max | INBRX-109 | |
| | EC₅₀ | Cytotox at | EC₅₀ | Max |
| Cell Line | (nM) | 125 nM (%) | (nM) | Cytotox (%) |
| CAL-78 | 145.5 | 10.9 | 114.6 | 194 |
| OUMS-27 | 109.1 | 24.0 | 118.2 | 87.8 |
| HS-SY-II | 108.4 | 55.8 | 102.6 | 95.1 |
| SW1353 | 201.8 | 21.7 | 133.4 | 88.3 |
| H-EMC-SS | 114.4 | 21.9 | 53.94 | 99 |
| SW620 | Unstable | 21.7 | 50.20 | 98.7 |

Table 11 shows the EC50 values of the INBRX-109 titration curves with and without 125 nM enitociclib. Maximum cytotoxicity was calculated by subtracting the % survival from 100.

TABLE 11

| | | | INBRX-109 + 125 nM | |
|---|---|---|---|---|
| | INBRX-109 Only | | Enitociclib | |
| | EC₅₀ | Max Cytotox (%) | EC₅₀ | Max |
| Cell Line | (pM) | at 1 nM | (pM) | Cytotox (%) |
| CAL-78 | 28.95 | 11.2 | 0.68 | 72.6 |
| OUMS-27 | Unstable | ~0 | 0.93 | 56.5 |
| HS-SY-II | 11.02 | 7.4 | 646.0 | 71.0 |
| SW1353 | 9.1 | 8.8 | 1.05 | 45.1 |
| H-EMC-SS | 9.26 | 95.8 | 2.33 | 98.2 |
| SW620 | 1.98 | 10.6 | 0.67 | 93.0 |

Table 12 shows the EC50 values of the enitociclib titration curve with and without 1 nM INBRX-109. Maximum cytotoxicity was calculated by subtracting the % survival from 100.

TABLE 12

| | | | Enitociclib + 1 nM | |
|---|---|---|---|---|
| | Enitociclib Only | | INBRX-109 | |
| | EC₅₀ | Max Cytotox at | EC₅₀ | Max |
| Cell Line | (nM) | 125 nM (%) | (nM) | Cytotox (%) |
| CAL-78 | 144.2 | 10.9 | 108.5 | 90.8 |
| OUMS-27 | 51.00 | 25.6 | 90.36 | 81 |
| HS-SY-II | 73.95 | 73.2 | 89.10 | 94 |
| SW1353 | 102.2 | 28.0 | 102.7 | 64.6 |
| H-EMC-SS | 123.6 | 15.8 | 113.8 | 99.2 |
| SW620 | 25.76 | 27.3 | 39.27 | 97.6 |

Table 13 shows the EC50 values of the INBRX-109 titration curves with and without 2 M AZD-5991. Maximum cytotoxicity was calculated by subtracting the % survival from 100.

TABLE 13

| | INBRX-109 Only | | INBRX-109 + 2 μM | |
|---|---|---|---|---|
| | | Max | AZD-5991 | |
| | EC₅₀ | Cytotox (%) | EC₅₀ | Max |
| Cell Line | (pM) | at 1 nM | (pM) | Cytotox (%) |
| CAL-78 | 0.62 | 12.9 | 3.93 | 64.9 |
| OUMS-27 | 8872.0 | 7.0 | 8.71 | 8.2 |
| HS-SY-II | 0.83 | 27.5 | Unstable | 92.1 |
| SW1353 | Unstable | 6.6 | 5.33 | 27.7 |
| H-EMC-SS | 10.19 | 96.2 | 2.59 | 99.6 |
| SW620 | 9.84 | 9.9 | 5.78 | 23.2 |

Table 14 shows the EC50 values of the AZD-5991 titration curve with and without 1 nM INBRX-109. Maximum cytotoxicity was calculated by subtracting the % survival from 100.

TABLE 14

| | AZD-5991 Only | | AZD-5991 + 1 nM | |
|---|---|---|---|---|
| | | Max | INBRX-109 | |
| | EC₅₀ | Cytotox at | EC₅₀ | Max |
| Cell Line | (μM) | 2 μM (%) | (μM) | Cytotox (%) |
| CAL-78 | 0.084 | 37.2 | 0.096 | 64.9 |
| OUMS-27 | >5000 | ~0 | 0.015 | 13.3 |
| HS-SY-II | 0.04 | 87.5 | 0.13 | 88.6 |
| SW1353 | 10.12 | 0 | 0.58 | 30.8 |
| H-EMC-SS | 0.18 | 11.9 | 0.007 | 99.5 |
| SW620 | 285.7 | 7.6 | unstable | 64.1 |

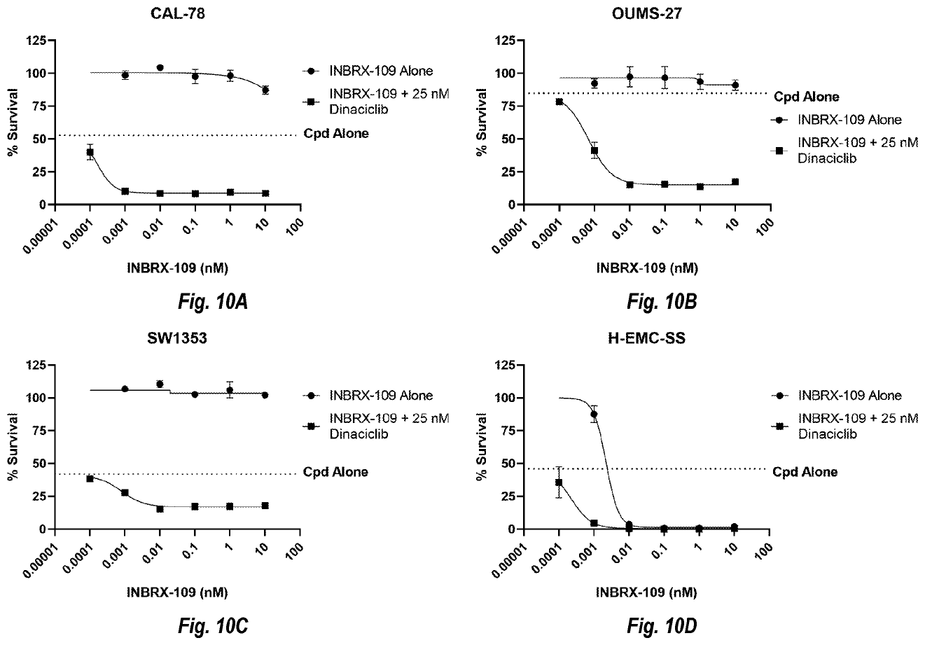

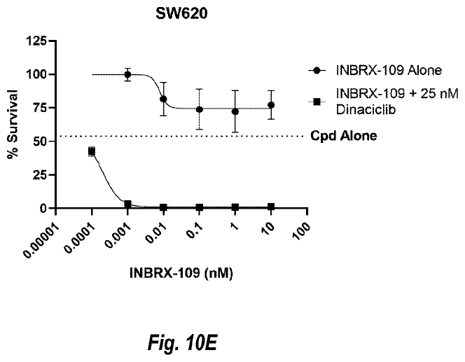

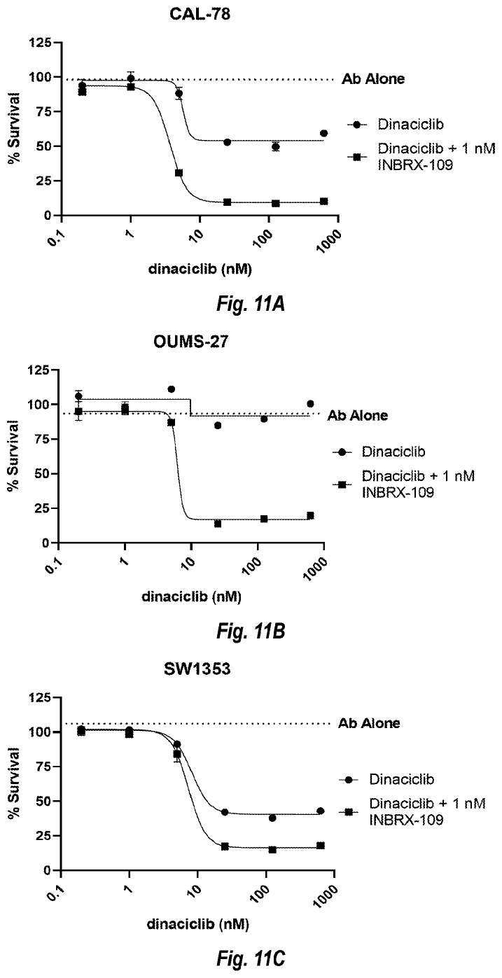

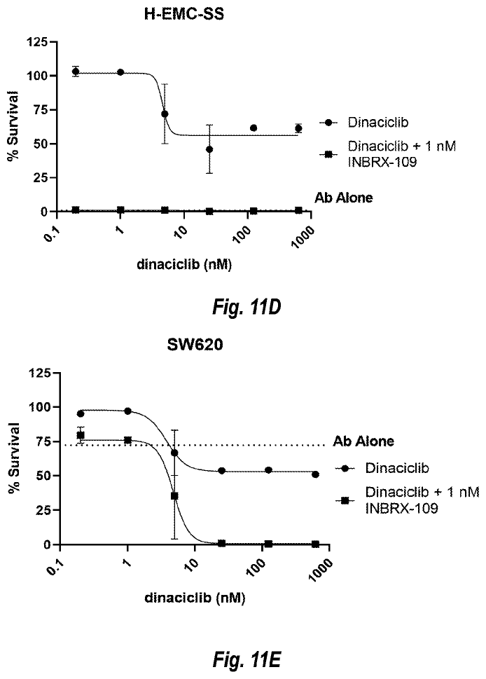

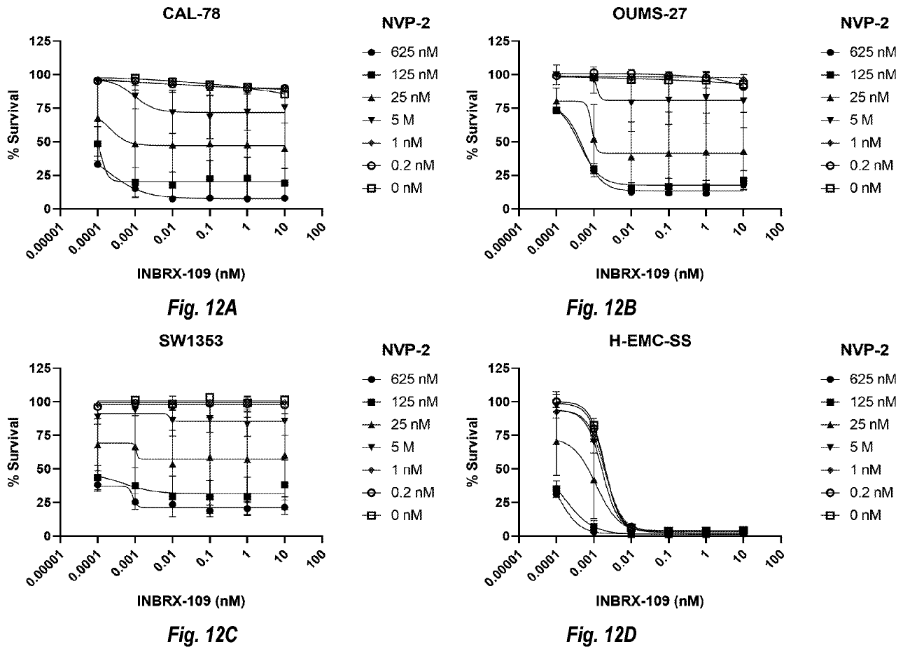

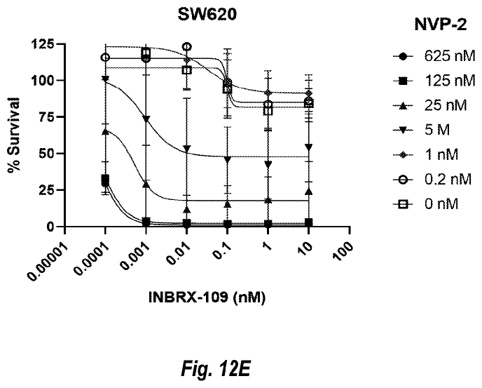

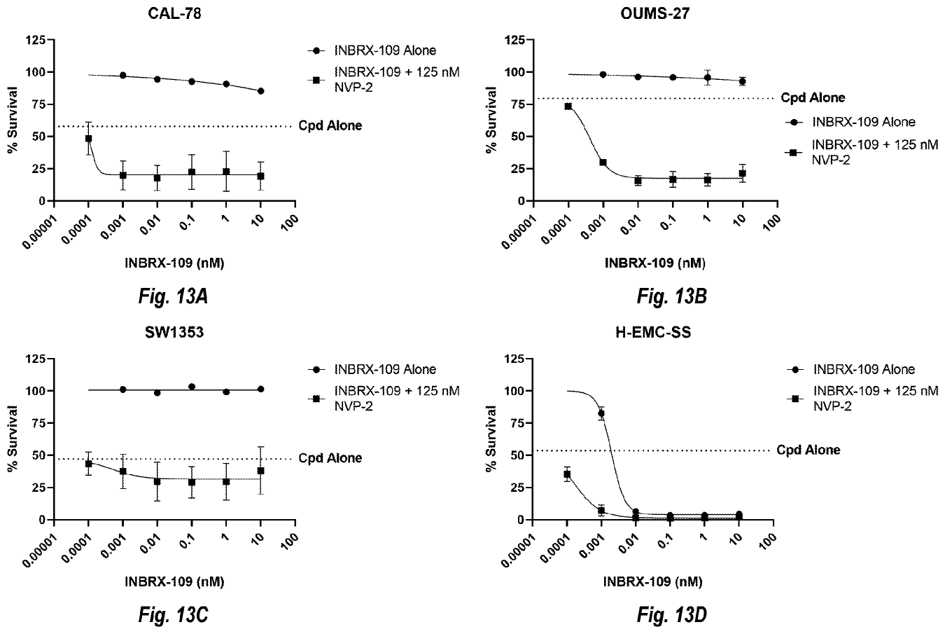

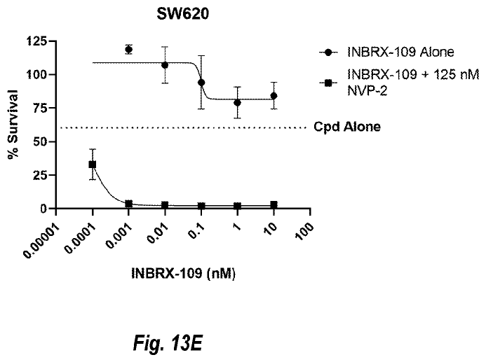

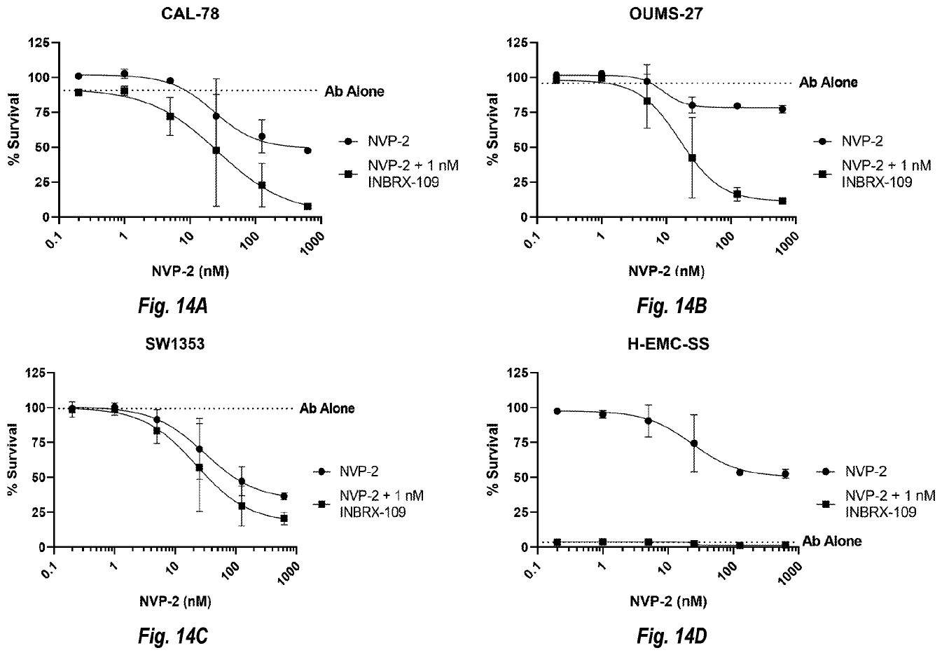

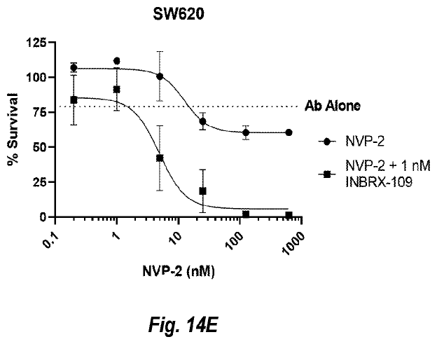

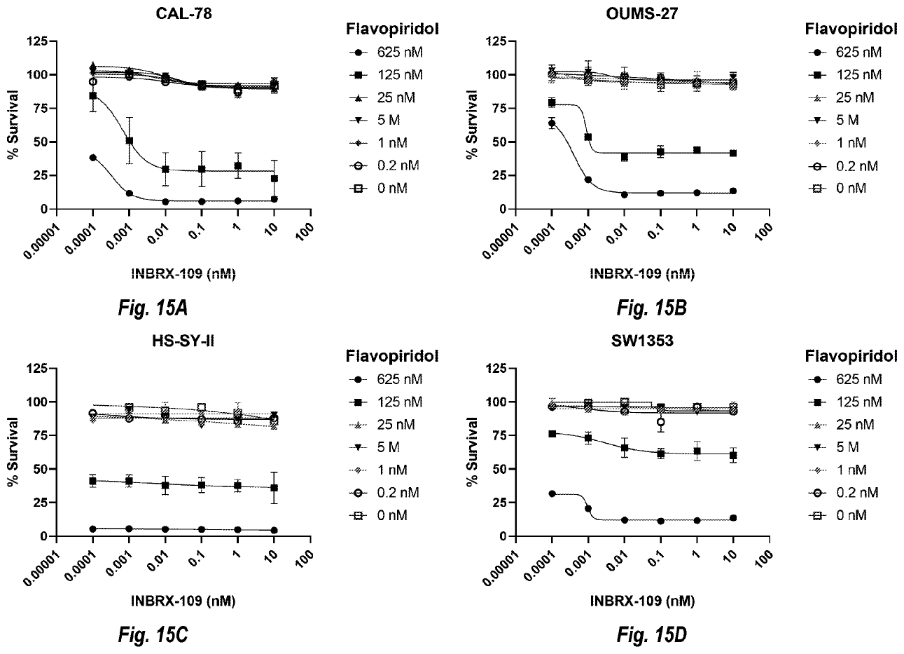

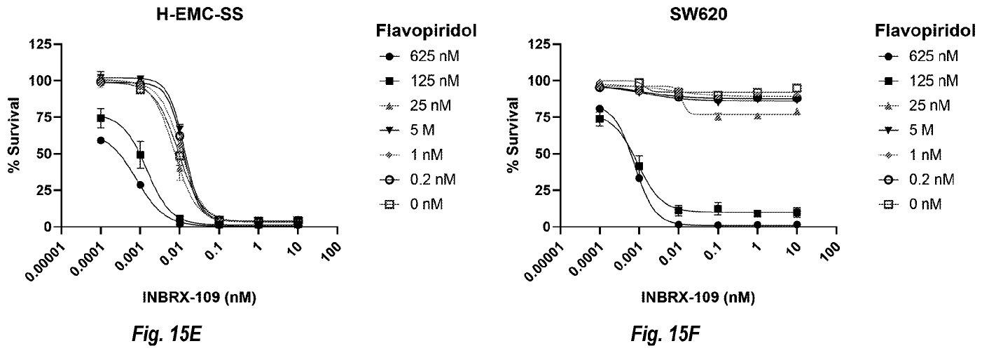

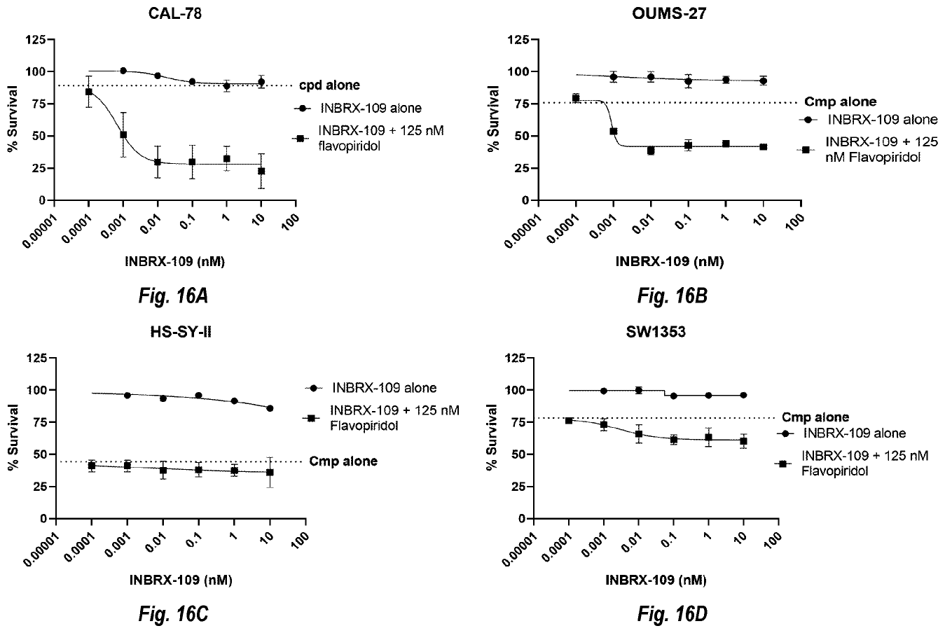

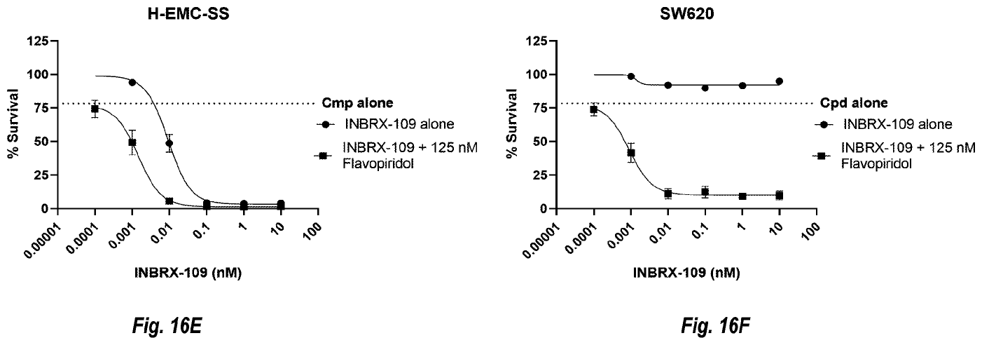

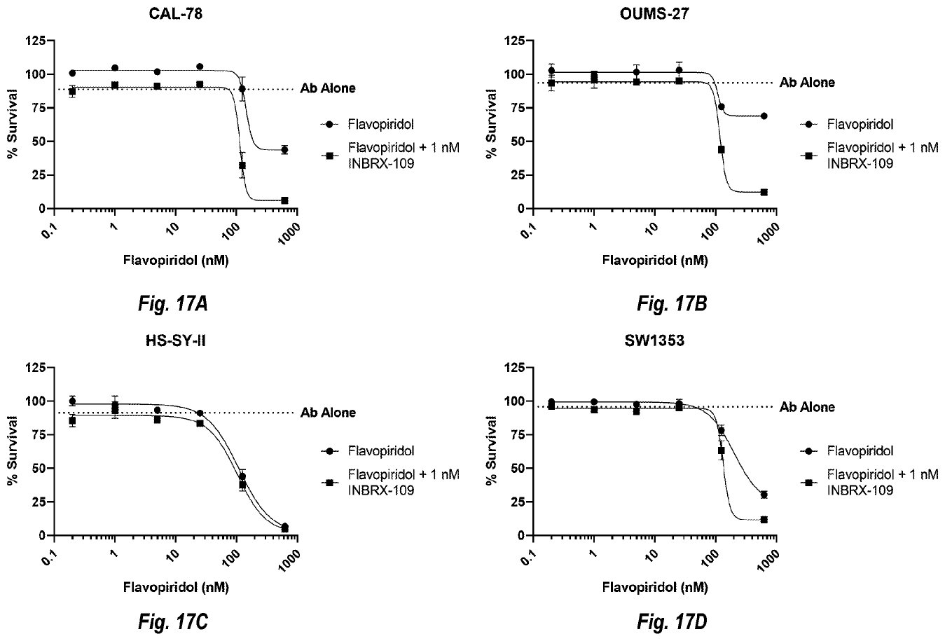

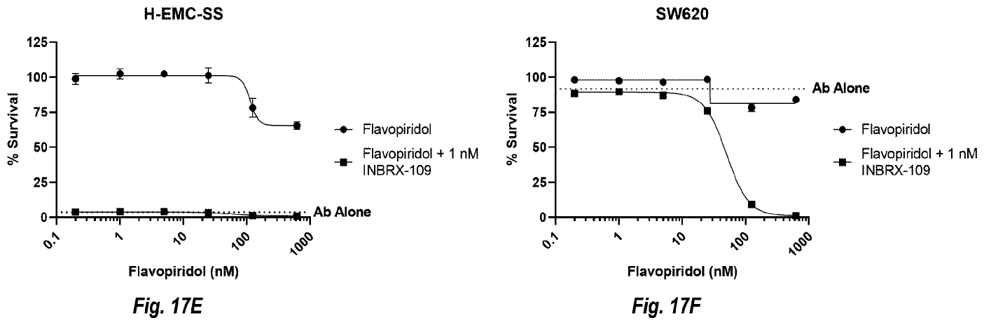

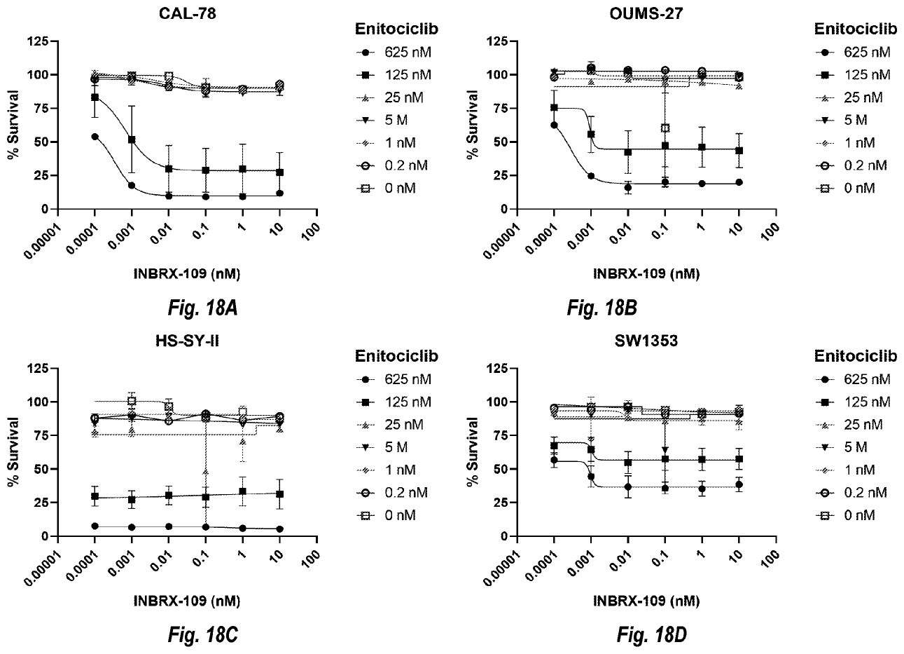

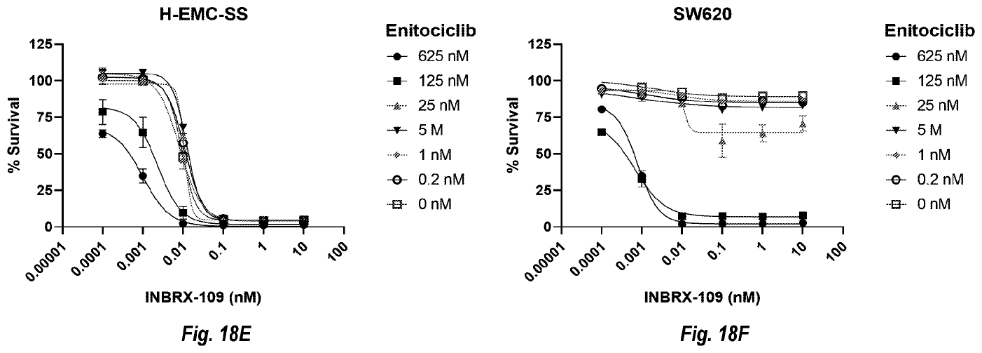

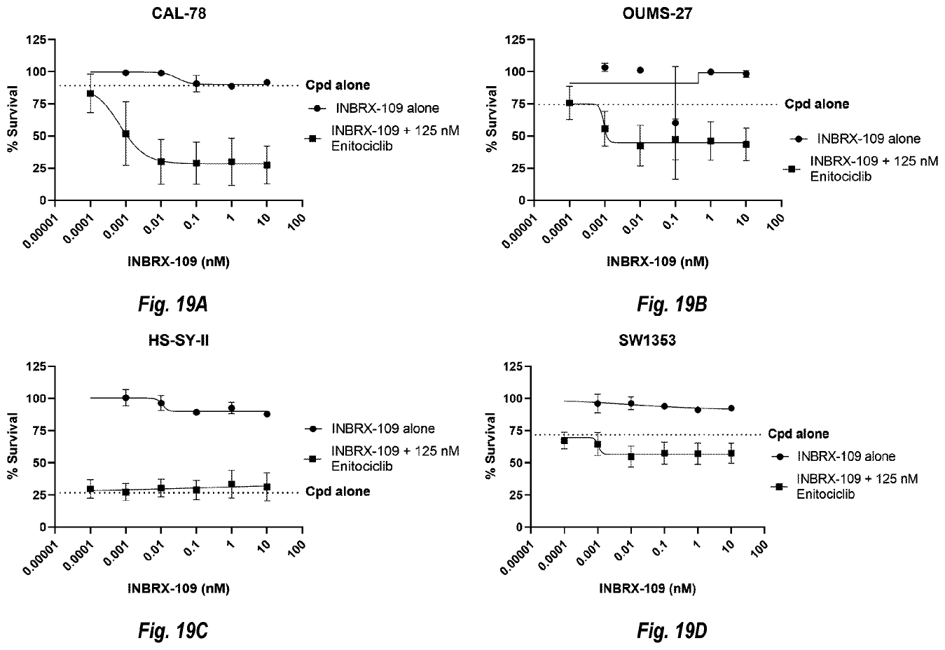

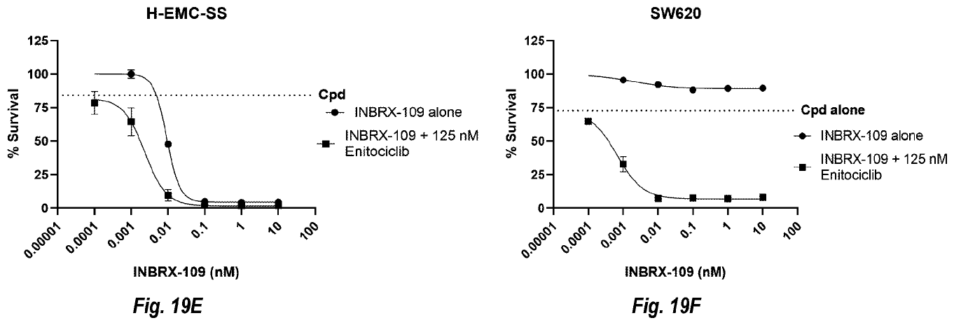

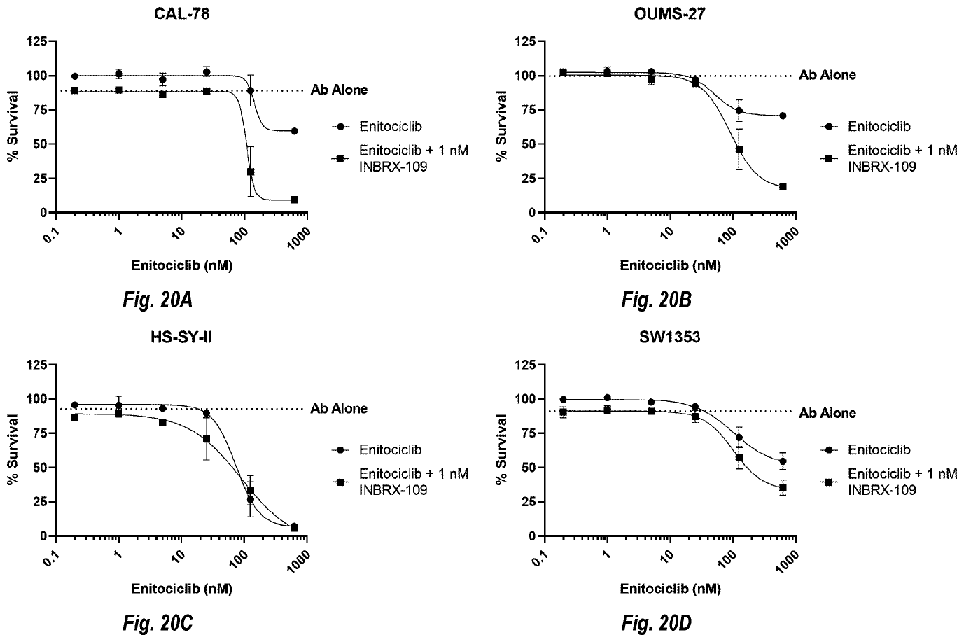

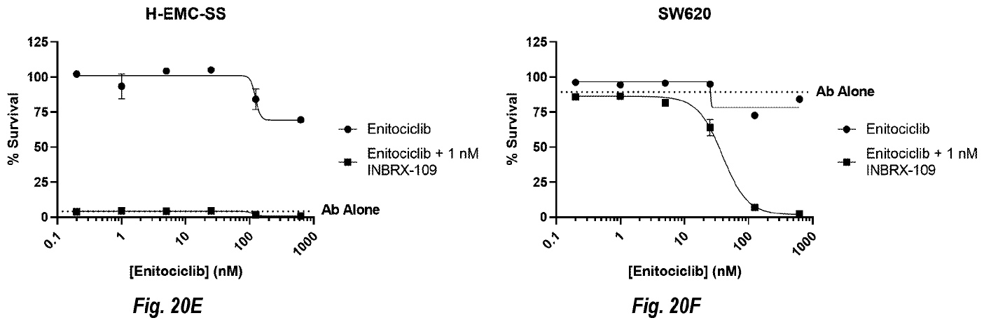

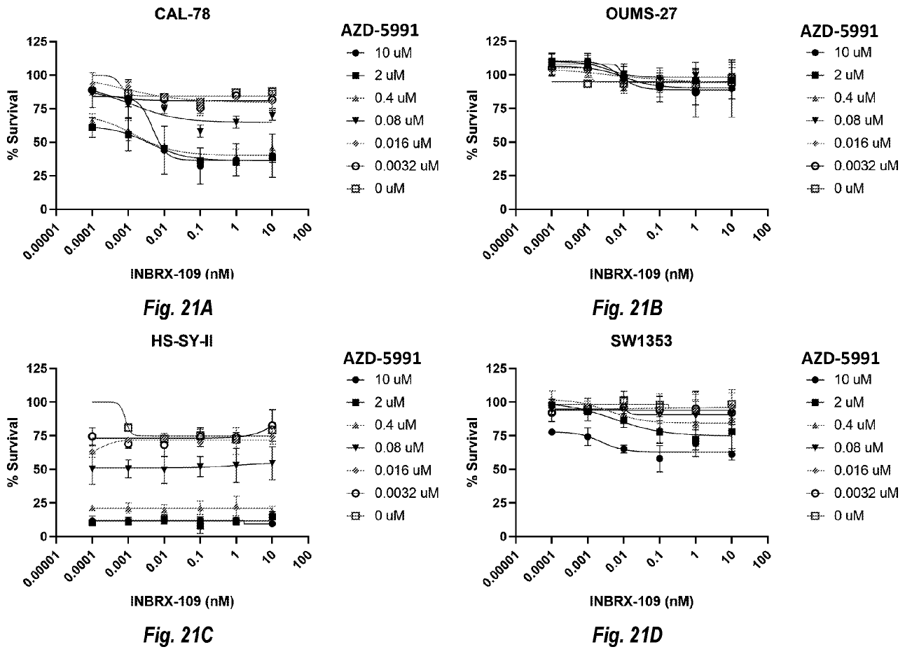

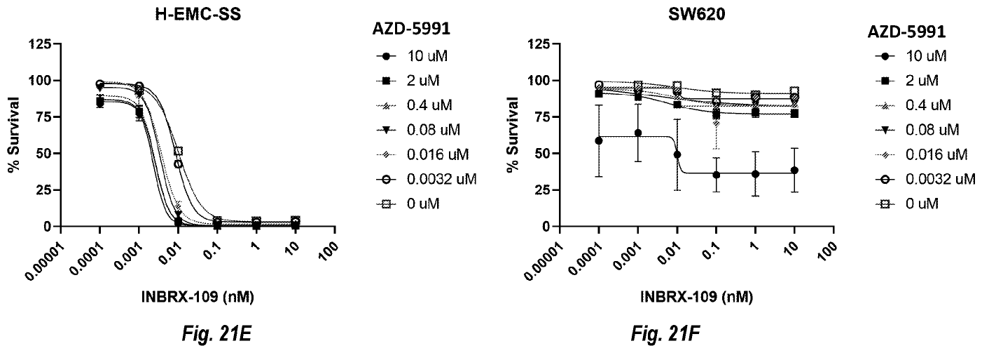

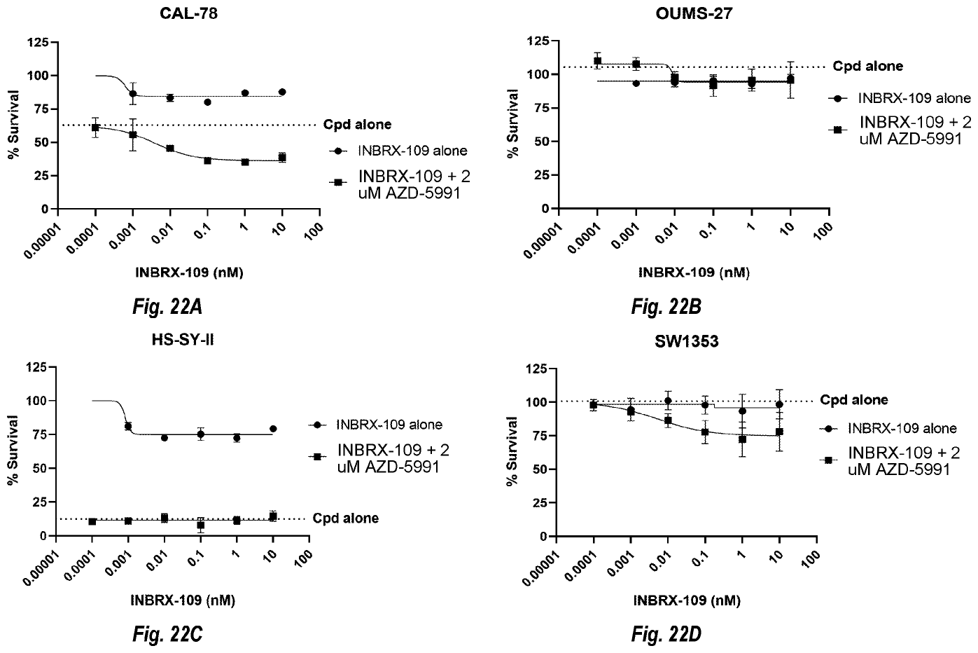

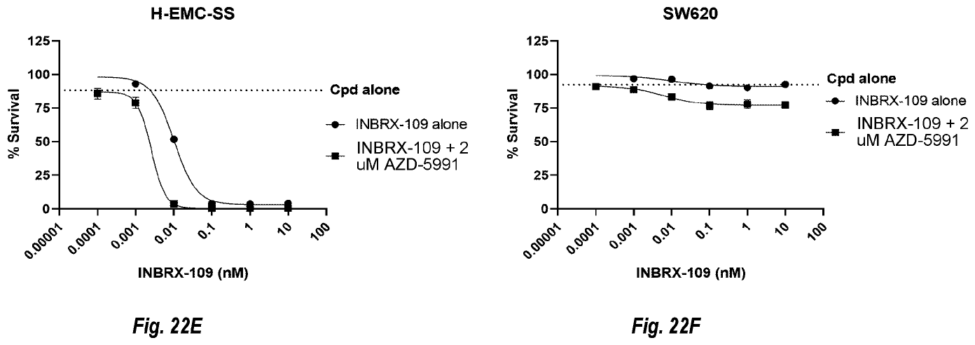

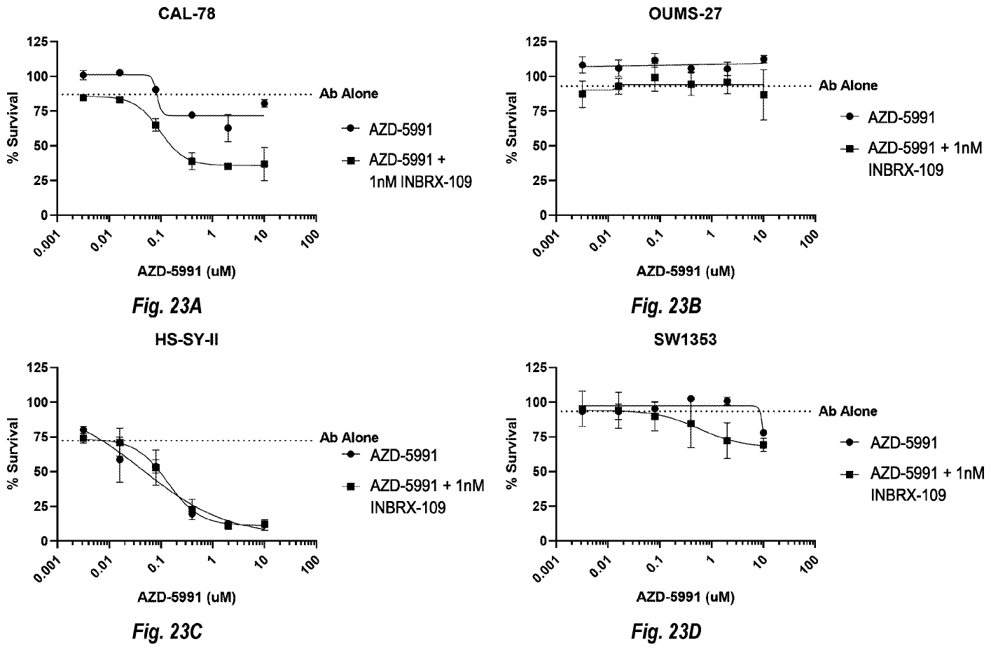

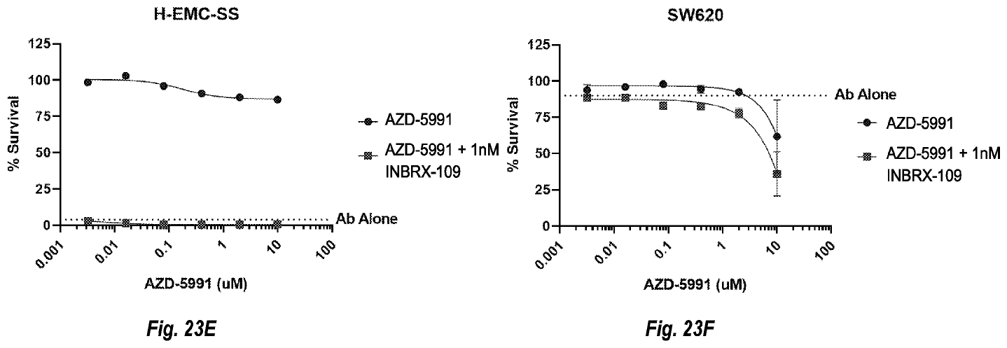

The combination of INBRX-109 with each of the tested CDK9 inhibitors, dinaciclib, NVP-2, flavopiridol, and enitociclib, showed increased cell killing on nearly every cancer cell line tested in these screens. The combination of NBRX-109 with AZD-5991, an inhibitor of MCL-1 (a target downstream of CDK9) also showed an increase in cell killing on a number of cell lines. Greater benefits were generally observed at concentrations higher than 25 nM for dinaciclib and NVP-2, at concentrations of 125 nM or higher for flavopiridol and enitociclib, and at concentrations higher than 0.4 M of AZD-5991. At these concentrations, the combination yielded a synergistic effect, as was apparent by the shift in the killing curves of INBRX-109 in combination with the CDK9, and downstream MCL-1 inhibitors as compared to the killing curves of INBRX-109 treatment alone. See, e.g., FIGS. 9-23 and Tables 5-14. Additionally, this combination led to a decrease in overall cell survival compared to treatment with either drug alone.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE of
Certain Sequences

| SEQ ID | | |
|---|---|---|
| NO | Description | Sequence |
| 1 | INBRX-109 | SGLTFPNYGM |
| | CDR1 | |
| 2 | INBRX-109 | AIYWSGGTVY |
| | CDR2 | |
| 3 | INBRX-109 | AVTIRGAATQTWKYDYW |
| | CDR3 | |
| 4 | INBRX-109 | EVQLLESGGGEVQPGGSLRLSCAASGLTFPNYGMGWFRQAPGKEREFVSAIYWS |
| | VHH | GGTVYYAESVKGRFTISRDNAKNTLYLQMSSLRAEDTAVYYCAVTIRGAATQTW |
| | | KYDYWGQGTLVTVKPGG |
| 5 | INBRX-109 | EVQLLESGGGEVQPGGSLRLSCAASGLTFPNYGMGWFRQAPGKEREFVSAIYWS |
| | VHH-linker- | GGTVYYAESVKGRFTISRDNAKNTLYLQMSSLRAEDTAVYYCAVTIRGAATQTW |
| | VHH | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGG |
| 6 | Fc (with | DKTHTCPPCPAPGGPSVFLFPPKPKDTLMISRTPEVTCVVVDVSHEDPEVKFNW |
| | hinge) | YVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQK |
| | | SLSLSPGK |
| 7 | INBRX-109 | EVQLLESGGGEVQPGGSLRLSCAASGLTFPNYGMGWFRQAPGKEREFVSAIYWS |
| | | GGTVYYAESVKGRFTISRDNAKNTLYLQMSSLRAEDTAVYYCAVTIRGAATQTW |
| | | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGGGGDKTHTCPPCPAPGG |
| | | PSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 8 | Human DR5 | MEQRGQNAPA ASGARKRHGP GPREARGARP GPRVPKTLVL VVAAVLLLVS |
| | (with signal | AESALITQQD LAPQQRAAPQ QKRSSPSEGL CPPGHHISED GRDCISCKYG |
| | peptide, | QDYSTHWNDL LFCLRCTRCD SGEVELSPCT TTRNTVCQCE EGTFREEDSP |
| | amino acids | EMCRKCRTGC PRGMVKVGDC TPWSDIECVH KESGTKHSGE VPAVEETVTS |
| | 1-55) | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CEDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKFMY LEGNADSAMS |
| 9 | Mature | ITQQD LAPQQRAAPQ QKRSSPSEGL CPPGHHISED GRDCISCKYG |
| | human DR5 | QDYSTHWNDL LFCLRCTRCD SGEVELSPCT TTRNTVCQCE EGTFREEDSP |
| | | EMCRKCRTGC PRGMVKVGDC TPWSDIECVH KESGTKHSGE VPAVEETVTS |
| | | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CFDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKFMY LEGNADSAMS |
| 10 | Human | MSAAVTAGKL ARAPADPGKA GVPGVAAPGA PAAAPPAKEI PEVLVDPRSR |
| | PLK1 | RRYVRGRFLG KGGFAKCFEI SDADTKEVFA GKIVPKSLLL KPHQREKMSM |
| | | EISIHRSLAH QHVVGFHGFF EDNDFVFVVL ELCRRRSLLE LHKRRKALTE |
| | | PEARYYLRQI VLGCQYLHRN RVIHRDLKLG NLFLNEDLEV KIGDFGLATK |
| | | VEYDGERKKT LCGTPNYIAP EVLSKKGHSF EVDVWSIGCI MYTLLVGKPP |
| | | FETSCLKETY LRIKKNEYSI PKHINPVAAS LIQKMLQTDP TARPTINELL |
| | | NDEFFTSGYI PARLPITCLT IPPRFSIAPS SLDPSNRKPL TVLNKGLENP |
| | | LPERPREKEE PVVRETGEVV DCHLSDMLQQ LHSVNASKPS ERGLVRQEEA |
| | | EDPACIPIFW VSKWVDYSDK YGLGYQLCDN SVGVLFNDST RLILYNDGDS |
| | | LQYIERDGTE SYLTVSSHPN SLMKKITLLK YFRNYMSEHL LKAGANITPR |
| | | EGDELARLPY LRTWFRTRSA IILHLSNGSV QINFFQDHTK LILCPLMAAV |
| | | TYIDEKRDFR TYRLSLLEEY GCCKELASRL RYARTMVDKL LSSRSASNRL |
| | | KAS |
| 11 | Human | MAKQYDSVECPFCDEVSKYEKLAKIGQGTFGEVFKARHRKTGQKVALKKVLMEN |
| | CDK9 | EKEGFPITALREIKILQLLKHENVVNLIEICRTKASPYNRCKGSIYLVFDFCEH |
| | | DLAGLLSNVLVKFTLSEIKRVMQMLLNGLYYIHRNKILHRDMKAANVLITRDGV |
| | | LKLADFGLARAFSLAKNSQPNRYTNRVVTLWYRPPELLLGERDYGPPIDLWGAG |
| | | CIMAEMWTRSPIMQGNTEQHQLALISQLCGSITPEVWPNVDNYELYEKLELVKG |
| | | QKRKVKDRLKAYVRDPYALDLIDKLLVLDPAQRIDSDDALNHDFFWSDPMPSDL |
| | | KGMLSTHLTSMFEYLAPPRRKGSQITQQSTNQSRNPATTNQTEFERVF |

What is claimed is:

1. A method of treating cancer in a subject in need thereof, comprising administering to the subject (a) a Death Receptor 5 (DR5) agonist, and (b) a Polo-Like Kinase 1 (PLK1) inhibitor.

2. The method of claim 1, wherein the DR5 agonist is INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), or MM201-b (Merrimack Pharmaceuticals).

3. The method of claim 2, wherein the DR5 agonist is INBRX-109.

4. The method of claim 1, wherein the DR5 agonist is a DR5-binding polypeptide.

5. The method of claim 4, wherein the DR5-binding polypeptide is at least tetravalent.

6. The method of any one of claims 3-5, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

7. The method of claim 6, wherein the at least one VHH domain comprises an amino acid sequence at least 90%, at least 95%, at least 99%, or 100% identical to the amino acid sequence of SEQ ID NO: 4.

8. The method of any one of claims 3-7, wherein the DR5-binding polypeptide comprises a VHH domain comprising the amino acid sequence of SEQ ID NO: 4.

9. The method of any one of claims 3-8, wherein the DR5-binding polypeptide comprises an Fc region.

10. The method of claim 9, wherein the Fc region comprises the amino acid sequence of SEQ ID NO: 6.

11. The method of any one of claims 3-10, wherein the DR5-binding polypeptide has the structure VHH-linker-VHH-linker-Fc.

12. The method of any one of claims 3-11, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

13. The method of any one of claims 3-12, wherein the VHH-linker-VHH comprises an amino acid sequence at least 90%, at least 95%, at least 99%, or 100% identical to the amino acid sequence of SEQ ID NO: 5.

14. The method of claim 13, wherein the VHH-linker-VHH comprises the amino acid sequence of SEQ ID NO: 5.

15. The method of any one of claims 3-14, wherein the DR5-binding polypeptide comprises an amino acid sequence at least 90%, at least 95%, at least 99%, or 100% identical to the amino acid sequence of SEQ ID NO: 7.

16. The method of any one of claims 3-15, wherein the DR5-binding polypeptide comprises the amino acid sequence of SEQ ID NO: 7.

17. The method of any one of claims 3-15, wherein the DR5-binding polypeptide consists of the amino acid sequence of SEQ ID NO: 7.

18. The method of any one of claims 1-17, wherein the PLK1 inhibitor is a small molecule or an interfering RNA (siRNA).

19. The method of any one of claims 1-18, wherein the PLK1 inhibitor is onvansertib, volasertib, rigosertib, BI2536 (Boehringer Ingelheim), N-[[4-[(6-Chloro-3-pyridinyl)methoxy]-3-methoxyphenyl]methyl]-3,4-dimethoxybenzeneethanamine hydrochloride (SBE 13 HCl), MLN0905 (Takeda Oncology), GSK461364 (GlaxosSmithKline), poloxin, poloxin-2HT, RO3280 (CAS No. 1062243-51-9), HMN-214 (CAS No. 173529-46-9), HMN-176 (CAS No. 173529-10-7), 2-cyano-2-[3-ethyl-4-oxo-5-[[3-(2-pyrrolidin-1-ylethyl)anilino]methyl]-1,3-thiazolidin-2-yl]-N-(2,2,2-trifluoroethyl)acetamide (ZK-thiazolidinone), or cyclapolin 9 (CAS No. 40533-25-3), 5-(5,6-Dimethoxy-1H-benzimidazol-1-yl)-3-[[2-(trifluoromethyl)phenyl]methoxy]-2-thiophenecarboxamide (GW 843682X).

20. The method of claim 19, wherein the PLK1 inhibitor is onvansertib, volasertib, rigosertib, BI2536 (Boehringer Ingelheim), MLN0905 (Takeda Oncology), GSK461364 (GlaxosSmithKline), CYC140 (Cyclacel), TKM-080301 (TKM-PLK1; Arbutus Biopharma), or TAK-960 (Takeda Pharmaceutical Company).

21. The method of any one of claims 1-18, wherein the PLK1 inhibitor is onvansertib.

22. The method of any one of claims 1-21, wherein the DR5 agonist and the PLK1 inhibitor are administered separately.

23. The method of claim 22, wherein the DR5 agonist and the PLK1 inhibitor are administered sequentially.

24. The method of claim 22 or 23, wherein at least one dose, or the first dose, of the DR5 agonist is administered before the PLK1 inhibitor.

25. The method of claim 22 or 23, wherein at least one dose, or the first dose, of the DR5 agonist is administered after the PLK1 inhibitor.

26. The method of any one of claims 1-21, wherein the DR5 agonist and the PLK1 inhibitor are administered concurrently.

27. The method of any one of claims 1-26, wherein the DR5 agonist and the PLK1 inhibitor act synergistically.

28. The method of claim 27, wherein synergy is determined in an in vitro cell survival assay.

29. The method of any one of claims 1-28, wherein administration of the DR5 agonist and the PLK1 inhibitor results in a synergistic effect, compared to each agent administered alone.

30. A method of treating cancer in a subject in need thereof, comprising administering to the subject (a) a Death Receptor 5 (DR5) agonist, and (b) a Cyclin-Dependent Kinase (CDK) inhibitor.

31. The method of claim 30, wherein the DR5 agonist is INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), or MM201-b (Merrimack Pharmaceuticals).

32. The method of claim 31, wherein the DR5 agonist is INBRX-109.

33. The method of claim 30, wherein the DR5 agonist is a DR5-binding polypeptide.

34. The method of claim 33, wherein the DR5-binding polypeptide is at least tetravalent.

35. The method of any one of claims 32-34, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

36. The method of claim 35, wherein the at least one VHH domain comprises an amino acid sequence at least 90%, at least 95%, at least 99%, or 100% identical to the amino acid sequence of SEQ ID NO: 4.

37. The method of any one of claims 32-36, wherein the DR5-binding polypeptide comprises a VHH domain comprising the amino acid sequence of SEQ ID NO: 4.

38. The method of any one of claims 32-37, wherein the DR5-binding polypeptide comprises an Fc region.

39. The method of claim 38, wherein the Fc region comprises the amino acid sequence of SEQ ID NO: 6.

40. The method of any one of claims 32-39, wherein the DR5-binding polypeptide has the structure VHH-linker-VHH-linker-Fc.

41. The method of any one of claims 32-40, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

42. The method of any one of claims 32-41, wherein the VHH-linker-VHH comprises an amino acid sequence at least 90%, at least 95%, at least 99%, or 100% identical to the amino acid sequence of SEQ ID NO: 5.

43. The method of claim 42, wherein the VHH-linker-VHH comprises the amino acid sequence of SEQ ID NO: 5.

44. The method of any one of claims 32-43, wherein the DR5-binding polypeptide comprises an amino acid sequence at least 90%, at least 95%, at least 99%, or 100% identical to the amino acid sequence of SEQ ID NO: 7.

45. The method of any one of claims 32-44, wherein the DR5-binding polypeptide comprises the amino acid sequence of SEQ ID NO: 7.

46. The method of any one of claims 32-44, wherein the DR5-binding polypeptide consists of the amino acid sequence of SEQ ID NO: 7.

47. The method of any one of claims 30-46, wherein the CDK inhibitor is a CDK9 inhibitor.

48. The method of any one of claims 30-47, wherein the CDK inhibitor is a small molecule.

49. The method of any one of claims 30-48, wherein the CDK inhibitor is flavopiridol, seliciclib, dinaciclib, atuveciclib, enitociclib, AZD4573, i-CDK9, or NVP-2.

50. The method of any one of claim 49, wherein the CDK inhibitor is dinaciclib, NVP-2, flavopiridol, enitociclib, or AZD4573.

51. The method of any one of claims 30-50, wherein the DR5 agonist and the CDK inhibitor are administered separately.

52. The method of claim 51, wherein the DR5 agonist and the CDK inhibitor are administered sequentially.

53. The method of claim 51 or 52, wherein at least one dose, or the first dose, of the DR5 agonist is administered before the CDK inhibitor.

54. The method of claim 51 or 52, wherein at least one dose, or the first dose, of the DR5 agonist is administered after the CDK inhibitor.

55. The method of any one of claims 30-51, wherein the DR5 agonist and the CDK inhibitor are administered concurrently.

56. The method of any one of claims 30-55, wherein the DR5 agonist and the CDK inhibitor act synergistically.

57. The method of claim 56, wherein synergy is determined in an in vitro cell survival assay.

58. The method of any one of claims 30-57, wherein administration of the DR5 agonist and the CDK inhibitor results in a synergistic effect, compared to each agent administered alone.

59. The method of any one of claims 1-58, wherein the cancer is adrenal cancer; astrocytoma; basal cell carcinoma, biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and/or pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

60. A DR5 agonist for use in a method of treating cancer, wherein the method comprises administering the DR5 agonist in combination with a PLK1 inhibitor.

61. Use of a DR5 agonist for the manufacture of a medicament for treating cancer, wherein the medicament is for administration with a PLK1 inhibitor.

62. A DR5 agonist for use in a method of treating cancer, wherein the method comprises administering the DR5 agonist in combination with a CDK inhibitor, such as a CDK9 inhibitor.

63. Use of a DR5 agonist for the manufacture of a medicament for treating cancer, wherein the medicament is for administration with a CDK inhibitor, such as a CDK9 inhibitor.


SEQUENCE LISTING (11 sequences)

SEQ ID NO: 1 - protein, 10 aa, synthetic construct
  source 1..10: note = INBRX-109 CDR1
SGLTFPNYGM

SEQ ID NO: 2 - protein, 10 aa, synthetic construct
  source 1..10: note = INBRX-109 CDR2
AIYWSGGTVY

SEQ ID NO: 3 - protein, 17 aa, synthetic construct
  source 1..17: note = INBRX-109 CDR3
AVTIRGAATQTWKYDYW

SEQ ID NO: 4 - protein, 125 aa, synthetic construct
  source 1..125: note = INBRX-109 VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 5 - protein, 254 aa, synthetic construct
  source 1..254: note = INBRX-109 VHH-linker-VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 6 - protein, 224 aa, synthetic construct
  source 1..224: note = Fc (with hinge)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 480 aa, synthetic construct
  source 1..480: note = INBRX-109
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 440 aa, Homo sapiens
  source 1..440: note = Human DR5 (with signal peptide, amino acids 1-55)
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 9 - protein, 385 aa, Homo sapiens
  source 1..385: note = Mature human DR5
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 10 - protein, 603 aa, Homo sapiens
  source 1..603: note = Human PLK1
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 11 - protein, 372 aa, Homo sapiens
  source 1..372: note = Human CDK9
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF